(19) United States
(12) Patent Application Publication
(10) Pub. No.: US 2009/0047243 A1
(43) Pub. Date: Feb. 19, 2009

(54) COMBINATIONS FOR THE TREATMENT OF B-CELL PROLIFERATIVE DISORDERS

(76) Inventors: Richard Rickles, Arlington, MA (US); Laura Pierce, San Diego, CA (US); Margaret S. Lee, Middleton, MA (US)

Correspondence Address: CLARK & ELBING LLP, 101 FEDERAL STREET, BOSTON, MA, 02110, US

(21) Appl. No.: 12/175,121

(22) Filed: Jul. 17, 2008

Related U.S. Application Data
(60) Provisional application No. 60/959,877, filed on Jul. 17, 2007.
(60) Provisional application No. 60/965,595, filed on Aug. 21, 2007.

(51) Int. Cl. A61K 38/20; A61K 31/472; A61K 31/44; A61K 31/4015; A61K 31/7076; A61K 31/53; A61K 31/47; A61K 31/50; A61K 31/18; A61K 31/437; A61K 31/519; A61K 31/7105; A61P 35/00

(52) U.S. Cl. 424/85.2; 514/424; 514/46; 514/243; 514/312; 514/247; 514/605; 514/303; 514/267; 514/262.1

(57) ABSTRACT

The invention features compositions and methods employing combinations of an A2A receptor agonist and a PDE inhibitor for the treatment of a B-cell proliferative disorder, e.g., multiple myeloma.

### CROSS-REFERENCE TO RELATED APPLICATIONS

This application claims benefit of U.S. Provisional Application Nos. 60/959,877, filed Jul. 17, 2007, and 60/965,595, filed Aug. 21, 2007, each of which is hereby incorporated by reference.

### BACKGROUND OF THE INVENTION

The invention relates to the field of treatments for proliferative disorders.

Multiple Myeloma (MM) is a malignant disorder of antibody producing B-cells. MM cells flourish in the bone marrow microenvironment, generating tumors called plasmacytomas that disrupt haematopoesis and cause severe destruction of bone. Disease complications include anemia, infections, hypercalcemia, organ dysfunction and bone pain.

For many years, the combination of glucocorticoids (e.g., dexamethasone or prednisolone) and alkylating agents (e.g., melphalan) was standard treatment for MM, with glucocorticoids providing most of the clinical benefit. In recent years, treatment options have advanced with three drugs approved by the FDA-Velcade™ (bortezomib), thalidomide, and lenalidomide. Glucocorticoids remain the mainstay of treatment and are usually deployed in combination with FDA-approved or emerging drugs. Unfortunately, despite advances in the treatment, MM remains an incurable disease with most patients eventually succumbing to the cancer.

### SUMMARY OF THE INVENTION

In general, the invention features methods and compositions employing an A2A receptor agonist and a PDE inhibitor for the treatment of a B-cell proliferative disorder.

In one aspect, the invention features a method of treating a B-cell proliferative disorder by administering to a patient a combination of an A2A receptor agonist and a PDE inhibitor in amounts that together are effective to treat the B-cell proliferative disorder. Exemplary A2A receptor agonists, e.g., IB-MECA, Cl-IB-MECA, CGS-21680, regadenoson, apadenoson, binodenoson, BVT-115959, and UK-432097, are listed in Tables 1 and 2. Exemplary PDE inhibitors, e.g., trequinsin, zardaverine, roflumilast, rolipram, cilostazol, milrinone, papaverine, BAY 60-7550, or BRL-50481, are listed in Tables 3 and 4. In certain embodiments, the PDE inhibitor is active against PDE 4 or at least two of PDE 2, 3, 4, and 7. In other embodiments, the combination includes two or more PDE inhibitors that when combined are active against at least two of PDE 2, 3, 4, and 7. The A2A receptor agonist and PDE inhibitor may be administered simultaneously or within 28 days of one another.

Examples of B-cell proliferative disorders include autoimmune lymphoproliferative disease, B-cell chronic lymphocytic leukemia (CLL), B-cell prolymphocyte leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, indolent myeloma, smoldering myeloma, monoclonal gammopathy of unknown significance (MGUS), B-cell non-Hodgkin's lymphoma, small lymphocytic lymphoma, monoclonal immunoglobin deposition diseases, heavy chain diseases, mediastinal (thymic) large B-cell lymphoma, intravascular large B-cell lymphoma, primary effusion lymphoma, lymphomatoid granulomatosis, precursor B-lymphoblastic leukemia/lymphoma, Hodgkin's lymphoma (e.g., nodular lymphocyte predominant Hodgkin's lymphoma, classical Hodgkin's lymphoma, nodular sclerosis Hodgkin's lymphoma, mixed cellularity Hodgkin's lymphoma, lymphocyte-rich classical Hodgkin's lymphoma, and lymphocyte depleted Hodgkin's lymphoma), post-transplant lymphoproliferative disorder, and Waldenstrom's macroglobulineamia.

In other embodiments, the patient is not suffering from a comorbid immunoinflammatory disorder of the lungs (e.g., COPD or asthma) or other immunoinflammatory disorder, or the patient has been diagnosed with a B-cell proliferative disorder prior to commencement of treatment.

The method may further include administering an antiproliferative compound or combination of antiproliferative compounds, e.g., selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors (for example, NPI-0052), CD40 inhibitors, anti-CSI antibodies, FGFR3 inhibitors, VEGF inhibitors, MEK inhibitors, cyclin D1 inhibitors, NF-kB inhibitors, anthracyclines, histone deacetylases, kinesin inhibitors, phosphatase inhibitors, COX2 inhibitors, mTOR inhibitors, calcineurin antagonists, and IMiDs. Specific antiproliferative compounds and combinations thereof are provided herein, e.g., in Tables 5 and 6.

The method may also further include administering IL-6 to the patient. If not by direct administration of IL-6, patients may be treated with agent(s) to increase the expression or activity of IL-6. Such agents may include other cytokines (e.g., IL-1 or TNF), soluble IL-6 receptor a (sIL-6R α), platelet-derived growth factor, prostaglandin E1, forskolin, cholera toxin, dibutyryl cAMP, or IL-6 receptor agonists, e.g., the agonist antibody MT-18, K-7/D-6, and compounds disclosed in U.S. Pat. Nos. 5,914,106, 5,506,107, and 5,891,998.

The invention further features kits including a PDE inhibitor and an A2A receptor agonist in an amount effective to treat a B-cell proliferative disorder. Exemplary PDE inhibitors and A2A receptors are described herein. In certain embodiments, the PDE inhibitor has activity against at least two of PDE 2, 3, 4, and 7, or the kit includes two or more PDE inhibitors that when combined have activity against at least two of PDE 2, 3, 4, and 7. A kit may also include an antiproliferative compound or combination of antiproliferative compounds, e.g., selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors (for example, NPI-0052), CD40 inhibitors, anti-CSI antibodies, FGFR3 inhibitors, VEGF inhibitors, MEK inhibitors, cyclin D1 inhibitors, NF-kB inhibitors, anthracyclines, histone deacetylases, kinesin inhibitors, phosphatase inhibitors, COX2 inhibitors, mTOR inhibitors, calcineurin antagonists, and IMiDs. Specific antiproliferative compounds and combinations thereof are provided herein. A kit may also include IL-6, a compound that increases IL-6 expression, or an IL-6 receptor agonist. Kits of the invention may further include instructions for administering the combination of agents for treatment of the B-cell proliferative disorder.

The invention also features a kit including an A2A receptor agonist and instructions for administering the A2A receptor agonist and a PDE inhibitor to treat a B-cell proliferative disorder. Alternatively, a kit may include a PDE inhibitor and instructions for administering said PDE inhibitor and an A2A receptor agonist to treat a B-cell proliferative disorder.

The invention additionally features pharmaceutical compositions including a PDE inhibitor and an A2A receptor agonist in an amount effective to treat a B-cell proliferative disorder and a pharmaceutically acceptable carrier. Exemplary PDE inhibitors and A2A receptors are described herein.

In certain embodiments, corticosteroids are specifically excluded from the methods, compositions, and kits of the invention. In other embodiments, e.g., for treating a B-cell proliferative disorder other than multiple myeloma, the following PDEs are specifically excluded from the methods, compositions, and kits of the invention: piclamilast, roflumilast, roflumilast-N-oxide, V-11294A, CI-1018, arofylline, AWD-12-281, AWD-12-343, atizoram, CDC-801, lirimilast, SCH-351591, cilomilast, CDC-998, D-4396, IC-485, CC-1088, and KW4490.

By “A2A receptor agonist” is meant any member of the class of compounds whose antiproliferative effect on MM.1S cells is reduced in the presence of an A2A-selective antagonist, e.g., SCH 58261. In certain embodiments, the antiproliferative effect of an A2A receptor agonist in MM.1S cells (used at a concentration equivalent to the Ki) is reduced by at least 10, 20, 30, 40, 50, 60, 70, 80, or 90% by an A2A antagonist used at a concentration of at least 10-fold higher than it's Ki (for example, SCH 58261 (Ki=5 nM) used at 78 nM)). An A2A receptor agonist may also retain at least 10, 20, 30, 40, 50, 60, 70, 80, 90, or 95% of its antiproliferative activity in MM.1S cells in the presence of an A1 receptor antagonist (e.g., DPCPX (89 nM)), an A2B receptor antagonist (e.g., MRS 1574 (89 nM)), an A3 receptor antagonist (e.g., MRS 1523 (87 nM)), or a combination thereof. In certain embodiments, the reduction of agonist-induced antiproliferative effect by an A2A antagonist will exceed that of an A1, A2B, or A3 antagonist. Exemplary A2A Receptor Agonists for use in the invention are described herein.

By “PDE inhibitor” is meant any member of the class of compounds having an IC₅₀ of 100 μM or lower concentration for a phosphodiesterase. In preferred embodiments, the IC₅₀ of a PDE inhibitor is 40, 20, 10 μM or lower concentration. In particular embodiments, a PDE inhibitor of the invention will have activity against PDE 2, 3, 4, or 7 or combinations thereof in cells of the B-type lineage. In preferred embodiments, a PDE inhibitor has activity against a particular type of PDE when it has an IC₅₀ of 40 μM, 20 μM, 10 μM, 5 μM, 1 μM, 100 nM, 10 nM, or lower concentration. When a PDE inhibitor is described herein as having activity against a particular type of PDE, the inhibitor may also have activity against other types, unless otherwise stated. Exemplary PDE inhibitors for use in the invention are described herein.

By “B-cell proliferative disorder” is meant any disease where there is a disruption of B-cell homeostasis leading to a pathologic increase in the number of B cells. A B-cell cancer is an example of a B-cell proliferative disorder. A B-cell cancer is a malignancy of cells derived from lymphoid stem cells and may represent any stage along the B-cell differentiation pathway. Examples of B-cell proliferative disorders are provided herein.

By “effective” is meant the amount or amounts of one or more compounds sufficient to treat a B-cell proliferative disorder in a clinically relevant manner. An effective amount of an active varies depending upon the manner of administration, the age, body weight, and general health of the patient. Ultimately, the prescribers will decide the appropriate amount and dosage regimen. Additionally, an effective amount can be that amount of compound in a combination of the invention that is safe and efficacious in the treatment of a patient having the B-cell proliferative disorder as determined and approved by a regulatory authority (such as the U.S. Food and Drug Administration).

By “treating” is meant administering or prescribing a pharmaceutical composition for the treatment or prevention of a B-cell proliferative disorder.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds. In certain embodiments, a patient is not suffering from a comorbid immunoinflammatory disorder.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; adrenocortical insufficiency; adrenogenital ayndrome; allergic conjunctivitis; allergic rhinitis; allergic intraocular inflammatory diseases, ANCA-associated small-vessel vasculitis; angioedema; ankylosing spondylitis; aphthous stomatitis; arthritis, asthma; atherosclerosis; atopic dermatitis; autoimmune disease; autoimmune hemolytic anemia; autoimmune hepatitis; Behcet's disease; Bell's palsy; berylliosis; bronchial asthma; bullous herpetiformis dermatitis; bullous pemphigoid; carditis; celiac disease; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; epicondylitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; hypersensitivity drug reactions; idiopathic cerato-scleritis; idiopathic pulmonary fibrosis; idiopathic thrombocytopenic purpura; inflammatory bowel or gastrointestinal disorders, inflammatory dermatoses; juvenile rheumatoid arthritis; laryngeal edema; lichen planus; Loeffler's syndrome; lupus nephritis; lupus vulgaris; lymphomatous tracheobronchitis; macular edema; multiple sclerosis; musculoskeletal and connective tissue disorder; myasthenia gravis; myositis; obstructive pulmonary disease; ocular inflammation; organ transplant rejection; osteoarthritis; pancreatitis; pemphigoid gestationis; pemphigus vulgaris; polyarteritis nodosa; polymyalgia rheumatica; primary adrenocortical insufficiency; primary billiary cirrhosis; pruritus scroti; pruritis/inflammation, psoriasis; psoriatic arthritis; Reiter's disease; relapsing polychondritis; rheumatic carditis; rheumatic fever; rheumatoid arthritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; serum sickness; shoulder tendinitis or bursitis; Sjogren's syndrome; Still's disease; stroke-induced brain cell death; Sweet's disease; systemic dernatomyositis; systemic lupus erythematosus; systemic sclerosis; Takayasu's arteritis; temporal arteritis; thyroiditis; toxic epidermal necrolysis; tuberculosis; type-1 diabetes; ulcerative colitis; uveitis; vasculitis; and Wegener's granulomatosis. “Non-dermal inflammatory disorders” include, for example, rheumatoid arthritis, inflammatory bowel disease, asthma, and chronic obstructive pulmonary disease. “Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis. By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis. As will be appreciated by one skilled in the art, a particular disease, disorder, or condition may be characterized as being both a proliferative skin disease and an inflammatory dermatosis. An example of such a disease is psoriasis.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition.

By a “high dosage” is meant at least 5% (e.g., at least 10%, 20%, 50%, 100%, 200%, or even 300%) more than the highest standard recommended dosage of a particular compound for treatment of any human disease or condition.

Compounds useful in the invention may also be isotopically labeled compounds. Useful isotopes include hydrogen, carbon, nitrogen, oxygen, phosphorous, fluorine, and chlorine, (e.g., ²H, 3H¹³C, ¹⁴C, ¹⁵N, 180, 170, ³¹P, ³²P, ³⁵S ¹⁸F, and ³⁶Cl). Isotopically-labeled compounds can be prepared by synthesizing a compound using a readily available isotopically-labeled reagent in place of a non-isotopically-labeled reagent.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein.

Other features and advantages of the invention will be apparent from the following detailed description, and from the claims.

### DETAILED DESCRIPTION OF THE INVENTION

The invention features methods, compositions, and kits for the administration of an effective amount of a combination of an A2A receptor agonist and a PDE inhibitor to treat a B-cell proliferative disorder. The invention is described in greater detail below.

### A2A Receptor Agonists

Exemplary A2A receptor agonists for use in the invention are shown in Table 1.

TABLE 1

| Compound | Synonym |
|---|---|
| (S)-ENBA | S-N⁶-(2-endo-norbornyl)adenosine |
| 2-Cl-IB-MECA | 2-chloro-N⁶-(3-iodobenzyl)-5′-N- |
| | methylcarboxamidoadenosine |
| ADAC | N-(4-(2-((4-(2-((2-aminoethyl)amino)-2- |
| | oxoethyl)phenyl)amino)-2-oxoethyl)phenyl)- |
| | Adenosine |
| AMP 579 | 1S-[1a,2b,3b,4a(S*)]-4-[7-[[1-[(3-chloro-2- |
| | thienyl)methylpropyl]propyl-amino]-3H- |
| | imidazo[4,5-b] pyridyl-3-yl]-N-ethyl-2,3- |
| | dihydroxycyclopentane carboxamide |
| Apadenoson | trans-4-(3-(6-amino-9-(N-ethyl-.beta.-D- |
| | ribofuranuronamidosyl)-9H-purin-2-yl)-2- |
| | propynyl)-Cyclohexanecarboxylic acid methyl |
| | ester |
| Apaxifylline | (S)-3,7-dihydro-8-(3-oxocyclopentyl)-1,3- |
| | dipropyl-1H-purine-2,6-dione |
| APEC | 2-[(2-aminoethyl-aminocarbonylethyl) |
| | phenylethylamino]-5′-N-ethyl- |
| | carboxamidoadenosine |
| ATL-193 | acetic acid 4-{3-[6-amino-9-(5-ethylcarbamoyl- |
| | 3,4-dihydroxy-tetrahydro-furan-2-yl)-9H- |
| | purin-2-yl]-prop-2-ynyl}-cyclohexylmethyl |
| | ester |
| ATL2037 | 5-{6-amino-2-[3-(4-hydroxymethyl-cyclohexyl)- |
| | prop-1-ynyl]-purin-9-yl}-3,4-dihydroxy- |
| | tetrahydro-furan-2-carboxylic acid ethylamide; |
| | BW-1433, 8-(4-carboxyethenylphenyl)-1,3- |
| | dipropylxanthine |
| ATL-313 | 4-{3-[6-amino-9-(5-cyclopropylcarbamoyl-3,4- |
| | dihydroxytetrahydrofuran-2-yl)-9H-purin-2- |
| | yl]prop-2-ynyl}piperidine-1-carboxylic acid |
| | methyl ester |
| ATL 210 | CAS Registry No.: 506438-25-1; |
| | WO 2003/029264 |
| BG 9928 | 1,3-dipropyl-8-[1-(4-propionate)-bicyclo- |
| | [2,2,2]octyl]xanthine |
| Binodenoson (MRE- | 2-((cyclohexylmethylene)hydrazino)-Adenosine |
| 0470) | |
| BN 063 | 1-cyclopropylisoguanosine |
| CCPA | 2-chloro-N⁶-cyclopentyladenosine |
| CDS 096370 | U.S. Pat. No. 6,800,633 |
| CGS 21680 | 2-(4-(2-carboxyethyl)phenethylamino)-5′-N- |
| | ethylcarboxamidoadenosine |
| CGS 21680c | 2-(4-(2-carboxyethyl)phenethylamino)-5′-N- |
| | ethylcarboxamidoadenosine, sodium salt |
| CGS 24012 | N6-2-(3,5-dimethoxyphenyl)-2-(2- |
| | methylphenyl)-ethyl adenosine |
| CHA | N⁶-cyclohexyladenosine |
| CP 608039 | (2S,3S,4R,5R)-3-amino-5-{6-[5-chloro-2-(3- |
| | methyl-isoxazol-5-ylmethoxy)-benzylamino]- |
| | purin-9-yl}-4-hydroxy-tetrahydro-furan-2- |
| | carboxylic acid methylamide |
| CPA | N⁶-cyclopentyladenosine |
| CPC 402 | 9′-hydroxy-EHNA |
| CPC 405 | 9′-chloro-EHNA |
| CPC 406 | 9′-phthalimido-EHNA |
| CPX | 1,3-dipropyl-8-cyclopentylxanthine |
| CV 1808 | 2-phenylaminoadenosine |
| CVT 2759 | [(5-{6-[((3R)oxolan-3-yl)amino]purin-9- |
| | yl}(3S,2R,4R,5R)-3,4-dihydroxyoxolan-2- |
| | yl)methoxy]-N-methylcarboxamide |
| CVT 3033 | (4S,2R,3R,5R)-2-[6-amino-2-(1-pentylpyrazol- |
| | 4-yl)purin-9-yl]-5-(-hydroxymethyl)oxolane- |
| | 3,4-diol |
| CVT 3619 | (2-{6-[((1R,2R)-2- |
| | hydroxycyclopentyl)amino]purin-9- |
| | yl}(4S,5S,2R,3R)-5-[(2-fluorophenylthio) |
| | methyl] oxolane-3,4-diol) |
| CVT 6883 | 3-ethyl-1-propyl-8-[1-(3-trifluoromethylbenzyl)- |
| | 1H-pyrazol-4-yl]-3,7-dihydropurine-2,6-dione |
| DAX | 1,3-diallyl-8-cyclohexylxanthine |
| DPCPX | 8-cyclopentyl-1,3-dipropylxanthine |
| DPMA | N⁶-(2-(3,5-dimethoxyphenyl)-2-(2- |
| | methylphenyl)ethyl)adenosine |
| FK 352 | (E)-(R)-1-[3-(2-phenylpyrazolo[1,5-a]pyridin-3- |
| | yl)acryloyl]pyperidin-2-ylacetic acid |
| FK 453 | (+)-(R)-[(E)-3-(2-phenylpyrazolo[1,5-a]pyridin- |
| | 3-yl) acryloyl]-2-piperidine ethanol |
| FK 838 | 6-oxo-3-(2-phenylpyrazolo [1,5-a] pyridin-3-yl)- |
| | 1(6H)-pyridazinebutanoic acid |
| GR 79236 | N-((1S,trans)-2-hydroxycyclopentyl)adenosine |
| HEMADO | 2-(1-hexynyl)-N-methyladenosine |
| HE-NECA | hexynyladenosine-5′-N-ethylcarboxamide |
| HPIA | N⁶-(R-4-hydroxyphenylisopropyl) adenosine |
| I-AB-MECA | N⁶-(4-amino-3-iodophenyl)methyl-5′-N- |
| | methylcarboxamidoadenosine |
| IB-MECA | N⁶-(3-iodobenzyl)-5′-N- |
| | methylcarboxamidoadenosine |
| IRFI 165 | 4-Cyclopentylamino-1.-methylimidazo[1,2- |
| | alquinoxaline |
| KF 17837 | (E)-8-(3,4-dimethoxystyryl)-1,3-dipropyl-7- |
| | methylxanthine |
| KF 20274 | 7,8-dihydro-8-ethyl-2-(3-noradamantyl)-4- |
| | propyl-1H-imidazo(2,1-j)purin-5(4H)-one |
| KF 21213 | (E)-8-(2,3-dimethyl-4-methoxystyryl)-1,3,7- |
| | trimethylxanthine |
| KFM 19 | 8-(3oxocyclopentyl)-1,3-dipropyl-7H-purine- |
| | 2,6-dione |
| KW 3902 | 8-(noradamantan-3-yl)-1,3-dipropylxanthine |
| MDL 102234 | 3,7-dihydro-8-(1-phenylpropyl)-1,3-dipropyl- |
| | 1H-purine-2,6-dione |
| MDL 102503 | (R)-3,7-dihydro-8-(1-methyl-2-phenylethyl)-1,3- |
| | dipropyl-1H-purine-2,6-dione |
| MDL 201449 | 9-[(1R,3R)-trans-cyclopentan-3-ol]adenine |
| Metrifudil | N-((2-methylphenyl)methyl)adenosine |
| Midaxifylline | 8-(1-Aminocyclopentyl)-3,7-dihydro-1,3- |
| | dipropyl-(1H)-purine-2,6-dione hydrochloride |
| Sonedenoson (MRE | 2-[2-(4-chlorophenyl)ethoxy]adenosine |
| 0094) | |
| N 0840 | N6-cyclopentyl-9-methyladenine |
| N 0861 | (+−)-N6-endonorbornan-2-yl-9-methyladenine |
| Naxifylline | 8-[(1S,2R,4S,5S,6S)-3- |
| | oxatricyclo[3.2.1.02,4]oct-6-yl]-1,3-dipropyl- |
| | 3,7-dihydro-1H-purine-2,6-dione |
| NECA | N-ethylcarboxamidoadenosine |
| PD 81723 | (2-Amino-4,5-dimethyl-3-thienyl)-[3- |
| | (trifluoromethyl)phenyl]methanone |
| Regadenoson (CVT | 2-(4-((methylamino)carbonyl)-1H-pyrazol-1-yl)- |
| 3146) | Adenosine |
| R-PIA | N-(1-methyl-2-phenylethyl)adenosine |
| SDZ WAG 994 | N⁶-cyclohexyl-2′-O-methyladenosine |
| SF 349 | 3-acetyl-7-methyl-7,8-dihydro-2,5(1H,6H) |
| | quinolinone |
| T 62 | (2-amino-4,5,6,7-tetrahydrobenzo[b]thiophen-3- |
| | yl)-(4-chlorophenyl)-methanone |
| TCPA | N⁶-cyclopentyl-2-(3- |
| | phenylaminocarbonyltriazene-1-yl)adenosine |
| UR 7247 | 3-iso-propyl-5-([2′-{1H}-tetrazol-5-yl-1,1′- |
| | biphenyl-4-yl]methyl)-1Hpyrazole-4- |
| | carboxylic acid |
| WRC 0342 | N⁶-(5′-endohydroxy)-endonorbornan-2-yl-9- |
| | methyladenine |
| WRC 0571 | C⁸-(N-methylisopropyl)-amino-N⁶(5′- |
| | endohydroxy)-endonorbornan-2-yl-9- |
| | methyladenine |
| YT 146 | 2-(1-octynyl) adenosine |
| ZM 241385 | 4-(2-[7-amino-2-(2-furyl)[1,2,4]-triazolo[2,3- |
| | a][1,3,5]triazin-5-yl amino]ethyl)phenol |
| Acadesine | 5-amino-1-[(2R,3R,4S,5R)-3,4-dihydroxy-5- |
| | (hydroxymethyl)oxolan-2-yl]imidazole-4- |
| | carboxamide |
| Capadenoson | 2-amino-6-({[2-(4-chlorophenyl)-1,3-thiazol-4- |
| | yl]methyl}sulfanyl)-4-[4-(2- |
| | hydroxyethoxy)phenyl]pyridine-3,5- |
| | dicarbonitrile |
| Spongosine | 2-methoxyadenosine |
| Adenogesic | Adenosine (intravenous) |
| Tocladesine | 8-chloro-cyclic adenosine monophosphate |
| APNEA | N⁶-2-(4-aminophenyl)ethyladenosine |
| CGS-15943 | 9-chloro-2-(2-furyl)-(1,2,4)triazolo(1,5- |
| | c)quinazolin-5-imine |
| CGS-22989 | 2-((2-(1-cyclohexen-1-yl)ethyl)amino)adenosine |
| GP-1-468 | 5-amino-5-deoxy-beta-D-ribofuranosylimidazole |
| | 4N-((4-chlorophenyl)methyl)carboxamide |
| GP-1-668 | 5-amino-1-beta-D-ribofuranosylimidazole 4N- |
| | ((4-nitrophenyl)methyl)carboxamide 5′- |
| | monophosphate |
| GP-531 | 5-amino-1-beta-D-(5′-benzylamino-5′- |
| | deoxyribofuranosyl)imidazole-4-carboxamide |
| LJ-529 | 2-chloro-N(6)-(3-iodobenzyl)-5′-N- |
| | methylcarbamoyl-4′-thioadenosine |
| NNC-21-0041 | 2-chloro-N-(1-phenoxy-2-propyl)adenosine |
| OT-7100 | 5-n-butyl-7-(3,4,5- |
| | trimethoxybenzoylamino)pyrazolo(1,5- |
| | a)pyrimidine |
| UP-202-32 | 1-(6-((2-(1-cyclopentylindol-3-yl)ethyl)amino)- |
| | 9H-purin-9-yl)-N-cyclopropyl-1-deoxy-beta-D- |
| | ribofuranuronamide |

Additional adenosine receptor agonists are shown in Table 2.

TABLE 2

| | | |
|---|---|---|
| 3′-Aminoadenosine-5′- | A15PROH | Adenosine |
| uronamides | | |
| Adenosine amine congener | Adenosine hemisulfate salt | BAY 68-4986 |
| solid | | |
| BIIB014 | BVT 115959 | CF 402 |
| CVT 2501 | DTI 0017 | GP 3367 |
| GP 3449 | GP 4012 | GR 190178 |
| GW 328267 | GW 493838 | Istradefylline |
| KF 17838 | M 216765 | MDL 101483 |
| NipentExtra | NNC 210113 | NNC 210136 |
| NNC 210147 | NNC 901515 | OSIC 113760 |
| SCH 420814 | SCH 442416 | SCH 59761 |
| Selodenoson (DTI-0009) | SLV 320 | SSR 161421 |
| SYN 115 | Tecadenoson (CVT-510) | UK 432097 |
| UP 20256 | WRC 0542 | Y 341 |
| BVT 115959 | UK 432097 | EPI-12323 c |
| GP-3269 | INO-7997 | INO-8875 |
| KS-341 | MEDR-440 | N-0723 |
| PJ-1165 | TGL-749 | Supravent |

Other adenosine receptor agonists are those described or claimed in Gao et al., JPET, 298: 209-218 (2001); U.S. Pat. Nos. 5,278,150, 5,424,297, 5,877,180, 6,232,297, 6,448,235, 6,514,949, 6,670,334, and 7,214,665; U.S. Patent Application Publication No. 20050261236, and International Publication Nos. WO98/08855, WO99/34804, WO2006/015357, WO2005/107463, WO03/029264, WO2006/023272, WO00/78774, WO2006/028618, WO03/086408, and WO2005/097140, incorporated herein by reference.

### PDE Inhibitors

Exemplary PDE inhibitors for use in the invention are shown in Table 3.

TABLE 3

| | | PDE |
|---|---|---|
| Compound | Synonym | Activity |
| 349U85 | 6-piperidino-2(1H)-quinolinone | 3 |
| Adibendan | 5,7-dihydro-7,7-dimethyl-2-(4-pyridinyl)- | 3 |
| | pyrrolo(2,3-f)benzimidazol-6(1H)-one | |
| Amlexanox | 2-amino-7-isopropyl-5-oxo-5H- | 3, 4 |
| | [1]benzopyrano[2,3-b]pyridine-3-carboxylic acid | |
| | (U.S. Pat. No. 4,143,042) | |
| Amrinone | 5-amino-(3,4′-bipyridin)-6(1H)-one | 3, 4 |
| Anagrelide | U.S. Pat. No. 3,932,407 | 3, 4 |
| AP 155 | 2-(1-piperazinyl)-4H-pyrido[1,2-a]pyrimidin-4- | 4 |
| | one | |
| AR 12456 | CAS Reg. No. 100557-06-0 | 4 |
| Arofylline | 3-(4-chlorophenyl)-3,7-dihydro-1-propyl-1H- | 4 |
| | purine-2,6-dione | |
| Ataquimast | 1-ethyl-3-(methylamino)-2(1H)-quinoxalinone | 3 |
| Atizoram | tetrahydro-5-[4-methoxy-3-[(1S,2S,4R)-2- | 4 |
| | norbornyloxy]phenyl]- | |
| | 2(1H)-pyrimidinone | |
| ATZ 1993 | 3-carboxy-4,5-dihydro-1-[1-(3- | |
| | ethoxyphenyl)propyl]-7-(5- | |
| | pyrimidinyl)methoxy-[1H]-benz[g]indazole | |
| | (Teikoku Hormone) | |
| Avanafil | 4-{[(3-chloro-4-methoxyphenyl)methyl]amino}- | 5 |
| | 2-[(2S)-2- | |
| | (hydroxymethyl)pyrrolidin-1-yl]-N-(pyrimidin- | |
| | 2-ylmethyl)pyrimidine- | |
| | 5-carboxamide | |
| AVE 8112 | | 4 |
| AWD 12171 | | 5 |
| AWD 12187 | | 7 |
| AWD 12250 | | 5 |
| AWD12343 | | 4 |
| BAY 38-3045 | | 1 |
| BAY 60-7550 (Alexis | 2-(3,4-dimethoxybenzyl)-7-[(1R)-1-[(1R)-1- | 2 |
| Biochemicals) | hydroxyethyl]-4-phenylbutyl]-5- | |
| | methylimidazo[5,1-f][1,2,4]triazin-4(3H)-one | |
| BBB 022 | | 4 |
| Bemarinone | 5,6-dimethoxy-4-methyl-2(1H)-quinazolinone | 3 |
| Bemoradan | 6-(3,4-dihydo-3-oxo-1,4(2H)-benzoxazin-7-yl)- | 3 |
| | 2,3,4,5-tetrahydro-5-methylpyridazin-3-one | |
| Benafentrine | (6-(p-acetamidophenyl)-1,2,3,4,4a,10b- | 3, 4 |
| | hexahydro-8,9-dimethoxy-2-methyl- | |
| | benzo[c][1,6]naphthyridine | |
| BMY 20844 | 1,3-dihydro-7,8-dimethyl-2H-imidazo[4,5- | 4 |
| | b]quinolin-2-one | |
| BMY 21190 | | 4 |
| BMY 43351 | 1-(cyclohexylmethyl)-4-(4-((2,3-dihydro-2-oxo- | 4 |
| | 1H-imidazo(4,5-b)quinolin-7-yl)oxy)-1- | |
| | oxobutyl)-Piperazine | |
| BRL 50481 | 3-(N,N-dimethylsulfonamido)-4-methyl- | 7 (7A) |
| | nitrobenzene | |
| C 3885 | | 4 |
| Caffeine citrate | 2-hydroxypropane-1,2,3-tricarboxylic acid | 4 |
| Apremilast (CC | N-(2-((1S)-1-(3-ethoxy-4-methoxyphenyl)-2- | 4 |
| 10004) | (methylsulfonyl)ethyl)-2,3-dihydro-1,3-dioxo- | |
| | 1H-isoindol-4-yl)-acetamide | |
| CC 1088 | | 4 |
| CC 3052 | The Journal of Immunology, 1998, 161: 4236- | 4 |
| | 4243 | |
| CC 7085 | | 4 |
| CCT 62 | 6-[(3-methylene-2-oxo-5-phenyl-5- | 3 |
| | tetrahydrofuranyl)methoxy]quinolinone | |
| CDC 998 | | 4 |
| CDP 840 | 4-((2R)-2-(3-(cyclopentyloxy)-4- | 4 |
| | methoxyphenyl)-2-phenylethyl)-pyridine | |
| CGH 2466 | 2-amino-4-(3,4-dichlorophenyl)-5-pyridin-4-yl- | 4 |
| | thiazol | |
| CI 1018 | N-(3,4,6,7-tetrahydro-9-methyl-4-oxo-1- | 4 |
| | phenylpyrrolo(3,2,1-jk)(1,4)benzodiazepin-3-yl)- | |
| | 4-pyridinecarboxamide | |
| CI 1044 | N-[9-amino-4-oxo-1-phenyl-3,4,6,7- | 4 |
| | tetrahydropyrrolo[3,2,1-jk][1,4]b-enzodiazepin- | |
| | 3(R)-yl]pyridine-3-carboxamide | |
| CI 930 | 4,5-dihydro-6-[4-(1H-imidazol-1-yl)phenyl]-5- | 3 |
| | methyl-3(2H)-pyridazinone | |
| Cilomilast (Ariflo ®) | 4-cyano-4-(3-cyclopentyloxy-4-methoxy- | 2, 3B, 4 |
| | phenyl)cyclohexane-1-carboxylic acid (U.S. | (4B, 4D) |
| | Pat. No. 5,552,438) | |
| Cilostamide | N-cyclohexyl-4-((1,2-dihydro-2-oxo-6- | 3 |
| | quinolinyl)oxy)-N-methyl-butanamide | |
| Cilostazol | 6-[4-(1-cyclohexyl-1H-tetrazol-5-yl)butoxy]-3,4- | 3, 4 |
| | dihydro-2(1H)-quinolinone (U.S. Pat. No. | |
| | 4,277,479) | |
| Cipamfylline | 8-amino-1,3-bis(cyclopropylmethyl)-3,7- | 4 |
| | dihydro-1H-purine-2,6-dione | |
| CK 3197 | 2H-imidazol-2-one, 1-benzoyl-5-(4-(4,5- | |
| | dihydro-2-methyl-1H-imidazol-1-yl)benzoyl)-4- | |
| | ethyl-1,3-dihydro | |
| CP 146523 | 4′-methoxy-3-methyl-3′-(5-phenyl-pentyloxy)- | 4 |
| | biphenyl-4-carboxylic acid | |
| CP 220629 | 1-cyclopentyl-3-ethyl-6-(2-methylphenyl)-7- | 4 |
| | oxo-4,5,6,7-tetrahydro-1H-pyrazolo[3,4- | |
| | c]pyridine | |
| CP 248 | (Z)-5-fluoro-2-methyl-1-[p- | 2 |
| | (methylsulfonyl)benzylidene]indene-3-acetic | |
| | acid | |
| CP 293121 | (S)-3-(3-cyclopentyloxy-4-methoxy)phenyl-2- | 4 |
| | isoxazoline-5-hydroxamic acid | |
| CP 353164 | 5-(3-cyclopentyloxy-4-methoxy-phenyl)- | 4 |
| | pyridine-2-carboxylic acid amide | |
| D 22888 | 8-methoxy-5-N-propyl-3-methyl-1-ethyl- | 4 |
| | imidazo [1,5-a]-pyrido [3,2-e]-pyrazinone | |
| D 4418 | N-(2,5-dichloro-3-pyridinyl)-8-methoxy-5- | 4 |
| | quinolinecarboxamide | |
| Dasantafil | 7-(3-bromo-4-methoxyphenylmethyl)-1-ethyl-8- | 5 |
| | {[(1R,2R)-2-hydroxycyclopentyl] = amino}-3- | |
| | (2-hydroxyethyl)-3,7-dihydro-1H-purine-2,6- | |
| | dione | |
| Dipyridamole | 2-{[9-(bis(2-hydroxyethyl)amino)-2,7-bis(1- | 5, 6, 7, 8, |
| | piperidyl)-3,5,8,10-tetrazabicyclo[4.4.0]deca- | 10, 11 |
| | 2,4,7,9,11-pentaen-4-yl]-(2- | |
| | hydroxyethyl)amino}ethanol | |
| DN 9693 | 1,5-dihydro-7-(1-piperidinyl)-imidazo[2,1- | 4 |
| | b]quinazolin-2(3H)-one dihydrochloride hydrate | |
| Doxofylline | 7-(1,3-dioxolan-2-ylmethyl)-1,3-dimethyl-3,7- | 4 |
| | dihydro-1H-purine-2,6-dione (U.S. Pat. No. | |
| | 4,187,308) | |
| E 4010 | 4-(3-chloro-4-metoxybenzyl)amino-1-(4- | 5 |
| | hydroxypiperidino)-6-phthalazinecarbonitrile | |
| | monohydrochloride | |
| B 4021 | sodium 1-[6-chloro-4-(3,4- | 4, 5 |
| | methylenedioxybenzyl)aminoquinazolin-2- | |
| | yl]piperidine-4-carboxylate sesquihydrate | |
| EHNA | erythro-9-(2-hydroxy-3-nonyl)adenine | 2, 3, 4 |
| EHT 0202 | 3,7-dimethyl-1-(5-oxohexyl)purine-2,6-dione | 4 |
| ELB 353 | | 4 |
| EMD 53998 | 5-(1-(3,4-dimethoxybenzoyl)-1,2,3,4-tetrahydro- | 3 |
| | 6-quinolyl)-6-methyl-3,6-dihydro-2H-1,3,4- | |
| | thiadiazin-2-one | |
| EMD 57033 | (+)-5-[1-(3,4-dimethoxybenzoyl)-3,4-dihydro- | 3 |
| | 2H-quinolin-6-yl]-6-methyl-3,6-dihydro-1,3,4- | |
| | thiadiazin-2-one | |
| EMD 57439 | (−)-5-[1-(3,4-dimethoxybenzoyl)-3,4-dihydro- | 3 |
| | 2H-quinolin-6-yl]-6-methyl-3,6-dihydro-1,3,4- | |
| | thiadiazin-2-one | |
| EMD 82639 | | 5 |
| EMR 62203 | | 5 |
| Enoximone | U.S. Pat. No. 4,405,635 | 3 |
| Enprofylline | 3-propyl xanthine | 4 |
| ER 017996 | 4-((3,4-(methylenedioxy)benzyl)amino)-6,7,8- | |
| | trimethoxyquinazoline | |
| Etazolate | 1-ethyl-4-((1-methylethylidene)hydrazino)-lh- | 4 |
| | pyrazolo(3,4-b) pyridine-5-carboxylic acid | |
| Exisulind | (1Z)-5-fluoro-2-methyl-1-[[4- | 2, 5 |
| | (methylsulfonyl)phenyl]methylene]-1H-indene- | |
| | 3-acetic acid | |
| Filaminast | (1E)-1-(3-(cyclopentyloxy)-4-methoxyphenyl)- | 4, 7 |
| | ethanone O-(aminocarbonyl)oxime | |
| FR 226807 | N-(3,4-dimethoxybenzyl)-2-{[(1R)-2-hydroxy-1- | 5 |
| | methylethyl]amino}-5-nitrobenzamide | |
| FR 229934 | | 5 |
| GI 104313 | 6-{4-[N-[-2-[3-(2-cyanophenoxy)-2- | 3 |
| | hydroxypropylamino]-2- | |
| | methylpropyl]carbamoylmethoxy-3- | |
| | chlorophenyl]}-4,5-dihydro-3(2H) pyridazinone | |
| GRC 3015 | | 4 |
| GSK 256066 | | 4 |
| GW 3600 | (7aS,7R)-7-(3-cyclopentyloxy-4- | 4 |
| | methoxyphenyl)-7a-methyl-2,5,6,7,7a-penta- | |
| | hydro-2-azapyrrolizin-3-one | |
| GW 842470 | N-(3,5-dichloro-4-pyridinyl)-1-((4- | 4 |
| | fluorophenyl)methyl)-5-hydroxy-α-oxo-1H- | |
| | indole-3-acetamide | |
| Helenalin | CAS Reg. No. 6754-13-8 | 5 |
| Hydroxypumafentrine | | 4 |
| IBMX | 3-isobutyl-1-methylxanthine | 3, 4, 5 |
| Ibudilast | 1-(2-isopropyl-pyrazolo[1,5-a]pyridine-3-yl)-2- | Not |
| | methylpropan-1-one (U.S. Pat. No. 3,850,941) | selective |
| IC 485 | | 4 |
| IPL 455903 | (3S,5S)-5-(3-cyclopentyloxy-4-methoxy- | 4 |
| | phenyl)-3-(3-methyl-benzyl)-piperidin-2-one | |
| Isbufylline | 1,3-dimethyl-7-isobutylxanthine | 4 |
| KF 17625 | 5-phenyl-1H-imidazo(4,5-c)(1,8)naphthyridin- | 4 |
| | 4(5H)-one | |
| KF 19514 | 5-phenyl-3-(3-pyridil) methyl-3H-imidazo[4,5- | 1, 4 |
| | c][1,8]naphthyridin-4(5H)-one | |
| KF 31327 | 3-ethyl-8-[2-[4-(hydroxymethyl)piperidin-1- | 5 |
| | yl]benzylamino]-2,3-dihydro-1H-imidazo[4,5- | |
| | g]quinazoline-2-thione | |
| Ks-505a | 1-carboxy- | 1 |
| | 2,3,4,4a,4b,5,6,6a,6b,7,8,8a,8b,9,10,10a, | |
| | 14,16,17,17a,17b,18,19,19a,19b, | |
| | 20,21,21a,21b,22,23,23a-dotriacontahydro-14- | |
| | hydroxy-8a,10a-bis(hydroxymethyl)-14-(3- | |
| | methoxy-3-oxopropyl)-1,4,4a,6,6a,17b,19b,21b- | |
| | octamethyl beta-D-glucopyranosiduronic acid | |
| KT 734 | | 5 |
| KW 4490 | | 4 |
| L 686398 | 9-[1,S,2R)-2-fluoro-1-methylpropyl]-2-methoxy- | 3, 4 |
| | 6-(1-piperazinyl]-purine hydrochloride | |
| L 826141 | 4-{2-(3,4-bis-difluromethoxyphenyl)-2-{4- | 4 |
| | (1,1,1,3,3,3-hexafluoro-2-hydroxypropan-2-yl)- | |
| | phenyl]-ethyl}-3-methylpyridine-1-oxide | |
| L 869298 | (+)-1 \| (S)-(+)-3-{2-[(3-cyclopropyloxy-4- | 4 |
| | difluromethoxy)-phenyl]-2-[5-(2-(1-hydroxy-1- | |
| | trifluoromethyl-2,2,2-trifluoro)ethyl)- | |
| | thiazolyl]ethyl}pyridine N-oxide | |
| L-869299 | (−)-1 \| (R)-(−)-3-{2-[(3-cyclopropyloxy-4- | 4 |
| | difluromethoxy)phenyl]-2-[5-(2-(1-hydroxy-1- | |
| | trifluoromethyl-2,2,2- | |
| | trifluoro)ethyl)thiazolyl]ethyl}pyridine N-Oxide | |
| Laprafylline | 8-[2-[4-(dicyclohexylmethyl)piperazin-1- | 4 |
| | yl]ethyl]-1-methyl-3-(2-methylpropyl)-7H- | |
| | purine-2,6-dione | |
| LAS 34179 | | 5 |
| LAS 37779 | | 4 |
| Levosimendan | U.S. Pat. No. 5,569,657 | 3 |
| Lirimilast | methanesulfonic acid 2-(2,4- | 4 |
| | dichlorophenylcarbonyl)-3-ureidobenzo-furan-6- | |
| | yl ester | |
| Lixazinone | N-cyclohexyl-N-methyl-4-((1,2,3,5-tetrahydro- | 3, 4 |
| | 2-oxoimidazo(2,1-b)quinazolin-7-yl)oxy)- | |
| | butanamide | |
| LPDE4 inhibitor | Bayer | 4 |
| Macquarimicin A | J Antibiot (Tokyo). 1995 Jun; 48 (6): 462-6 | |
| MEM 1414 | US 2005/0215573 A1 | 4 |
| MERCK1 | (5R)-6-(4-{[2-(3-iodobenzyl)-3-oxocyclohex-1- | 3 |
| | en-1-yl]amino}phenyl)-5-methyl-4,5- | |
| | dihydropyridazin-3(2H)-one; | |
| | dihydropyridazinone | |
| Mesopram | (5R)-5-(4-methoxy-3-propoxyphenyl)-5-methyl- | 4 |
| | 2-oxazolidinone | |
| Milrinone | 6-dihydro-2-methyl-6-oxo-3,4′-bipyridine)-5- | 3, 4 |
| | carbonitrile (U.S. Pat. No. 4,478,836) | |
| MIMX | 1 8-methoxymethyl-3-isobutyl-1-methylxantine | 1 |
| MN 001 | 4-[6-acetyl-3-[3-(4-acetyl-3-hydroxy-2- | 4 |
| | propylphenylthio)propoxy]-2- | |
| | propylphenoxy]butyric acid | |
| Mopidamol | U.S. Pat. No. 3,322,755 | 4 |
| MS 857 | 4-acetyl-1-methyl-7-(4-pyridyl)-5,6,7,8- | 3 |
| | tetrahydro-3(2H)-isoquinolinone | |
| Nanterinone | 6-(2,4-dimethyl-1H-imidazol-1-yl)-8-methyl- | 3 |
| | 2(1H)-quinolinone | |
| NCS 613 | J Pharmacol Exp Ther Boichot et al. 292 (2): | 4 |
| | 647 | |
| ND 1251 | | 4 |
| ND7001 | Neuro3D Pharmaceuticals | 2 |
| Nestifylline | 7-(1,3-dithiolan-2-ylmethyl)-1,3-dimethylpurine- | |
| | 2,6-dione | |
| NIK 616 | | 4 |
| NIP 520 | | 3 |
| NM 702 | | 5 |
| NSP 306 | | 3 |
| NSP 513 | | 3 |
| NSP 804 | 4,5-dihydro-6-[4-[(2-methyl-3-oxo-1- | 3 |
| | cyclopentenyl)-amino] phenyl]-3(2H)- | |
| | pyridazinone | |
| NSP 805 | 4,5-dihydro-5-methyl-6-[4-[(2-methyl-3-oxo-1- | 3 |
| | cyclopentenyl) amino]phenyl]-3(2H)- | |
| | pyridazinone | |
| NVP ABE 171 | | 4 |
| Oglemilast | N-(3,5-dichloropyridin-4-yl)-4-difluoromethoxy- | 4 |
| | 8-((methylsulfonyl)amino)dibenzo(b,d)furan-1- | |
| | carboxamide | |
| Olprinone | 5-imidazo[2,1-f]pyridin-6-yl-6-methyl-2-oxo- | 3, 4 |
| | 1H-pyridine-3-carbonitrile | |
| ONO 1505 | 4-[2-(2-hydroxyethoxy)ethylamino]-2-(1H- | 5 |
| | imidazol-1-yl)-6-methoxy-quinazoline | |
| | methanesulphonate | |
| ONO 6126 | | 4 |
| OPC 33509 | (−)-6-[3-[3-cyclopropyl-3-[(1R,2R)-2- | 3 |
| | hydroxyclohexyl]ureido]-propoxy]-2(1H)- | |
| | quinolinone | |
| OPC 33540 | 6-[3-[3-cyclooctyl-3-[(1R[*],2R[*])-2- | 3 |
| | hydroxycyclohexyl]ureido]-propoxy]-2(1H)- | |
| | quinolinone | |
| ORG 20241 | N-hydroxy-4-(3,4-dimethoxyphenyl)-thiazole-2- | 3, 4 |
| | carboximidamide | |
| ORG 30029 | N-hydroxy-5,6-dimethoxy-benzo[b]thiophene-2- | 3, 4 |
| | carboximide hydrochloride | |
| ORG 9731 | 4-fluoro-N-hydroxy-5,6-dimethoxy- | 3, 4 |
| | benzo[b]thiophene-2-carboximidamide | |
| | methanesulphonate | |
| ORG 9935 | 4,5-dihydro-6-(5,6-dimethoxy-benzo[b]-thien-2- | 3 |
| | yl)-methyl-1-(2H)-pyridazinone | |
| OSI 461 | N-benzyl-2-[(3Z)-6-fluoro-2-methyl-3-(pyridin- | 5 |
| | 4-ylmethylidene)inden-1-yl]acetamide | |
| | hydrochloride | |
| Osthole | 7-methoxy-8-(3-methyl-2-butenyl)-2H-1- | 5 |
| | benzopyran-2-one | |
| Ouazinone | (R)-6-chloro-1,5-dihydro-3-methyl-imidazo[2,1- | 3 |
| | b]quinazolin-2-one | |
| PAB 13 | 6-bromo-8-(methylamino)imidazo[1,2- | |
| | a]pyrazine | |
| PAB 15 | 6-bromo-8-(ethylamino)imidazo[1,2-a]pyrazine | |
| PAB 23 | 3-bromo-8-(methylamino)imidazo[1,2- | |
| | a]pyrazine | |
| Papaverine | 1-[(3.4-dimethoxyphenyl)-methyl]-6,7- | 5, 6, 7, 10 |
| | dimethoxyisoquinolone | |
| PDB 093 | | 4 |
| Pentoxifylline | 3,7-dimethyl-1-(5-oxohexyl)-3,7-dihydropurine- | |
| | 2,6-dione (U.S. Pat. No. 3,422,107) | |
| Piclamilast | 3-cyclopentyloxy-N-(3,5-dichloropyridin-4-yl)- | 2, 3B, 4 |
| | 4-methoxy-benzamide | (4B, 4D), 7 |
| Pimobendan | U.S. Pat. No. 4,361,563 | 3,4 |
| Piroximone | 4-ethyl-1,3-dihydro-5-(4-pyridinylcarbonyl)-2H- | 3 |
| | imidazol-2-one | |
| Prinoxodan | 6-(3,4-dihydro-3-methyl-2-oxoquinazolinyl)-4,5- | |
| | dihydro-3-pyridazinone | |
| Propentofylline | U.S. Pat. No. 4,289,776 | 5 |
| Pumafentrine | rel-(M)-4-((4aR,10bS)-9-ethoxy-1,2,3,4,4a,10b- | 3B, 4 (4B, |
| | hexahydro-8-methoxy-2-methylbenzo(c) | 4D) |
| | (1,6)naphthyridin-6-yl)-N,N-bis(1-methylethyl)- | |
| | benzamide | |
| R 79595 | N-cyclohexyl-N-methyl-2-[[[phenyl (1,2,3,5- | 3 |
| | tetrahydro-2 oxoimidazo [2,1-b]-quinazolin-7-yl) | |
| | methylene] amin] oxy] acetamide | |
| Revizinone | (E)-N-cyclohexyl-N-methyl-2-(((phenyl(1,2,3,5- | 3 |
| | tetrahydro-2-oxoimidazo(2,1-b)quinazolin-7- | |
| | yl)methylene)amino)oxy)-acetamide | |
| Ro20-1724 | 4-(3-butoxy-4-methoxybenzyl)-2- | 4 |
| | imidazolidinone | |
| Roflumilast | 3-(cyclopropylmethoxy)-N-(3,5-dichloro-4- | 2, 3B 4 (4B, |
| | pyridinyl)-4-(difluoromethoxy)-benzamide | 4D), 5 |
| Rolipram | 4-(3-cyclopentyloxy-4-methoxyphenyl)-2- | 4 |
| | pyrrolidone (U.S. Pat. No. 4,193,926) | |
| RPL554 | 9,10-dimethoxy-2(2,4,6-trimethylphenylimino)- | 3, 4 |
| | 3-(N-carbamoyl-2-aminoethyl)-3,4,6,7- | |
| | tetrahydro-2H-pyrimido[6,1-a]isoquinolin-4-one | |
| RPL565 | 6,7-dihydro-2-(2,6-diisopropylphenoxy)-9,10- | 3, 4 |
| | dimethoxy-4H-pyrimido[6,1-a]isoquinolin-4-one | |
| RPR 132294 | | 4 |
| RPR 132703 | | 4 |
| Saterinone | 1,2-dihydro-5-(4-(2-hydroxy-3-(4-(2- | 3 |
| | methoxyphenyl)-1-piperazinyl)propoxy)phenyl)- | |
| | 6-methyl-2-oxo-3-pyridinecarbonitrile | |
| Satigrel | 4-cyano-5,5-bis(4-methoxyphenyl)-4-pentenoic | 2, 3, 5 |
| | acid (U.S. Pat. No. 4,978,767) | |
| SCA 40 | 6-bromo-8-methylaminoimidazo[1,2- | 3 |
| | a]pyrazine-2carbonitrile | |
| SCH 351591 | N-(3,5-dichloro-1-oxido-4-pyridinyl)-8- | 4 |
| | methoxy-2-(trifluoromethyl)-5-quinoline | |
| | carboxamide | |
| SCH 45752 | J Antibiot (Tokyo). 1993 Feb; 46 (2): 207-13 | |
| SCH 46642 | | 5 |
| SCH 51866 | cis-5,6a,7,8,9,9a-hexahydro-2-(4- | 1, 5 |
| | (trifluoromethyl)phenylmethyl)-5-methyl- | |
| | cyclopent (4,5)imidazo(2,1-b)purin-4(3H)-one | |
| SCH 51866 | cis-5,6a,7,8,9,9a-hexahydro-2-[4- | 1, 5 |
| | (trifluoromethyl)phenylmethyl]-5-methyl- | |
| | cyclopent[4,5]imidazo[2,1-b]purin-4(3H)-one | |
| SCH 59498 | cis-2-hexyl-5-methyl-3,4,5,6a,7,8,9,9a- | 5 |
| | octahydrocyclopent[4,5]imidazo-[2,-1-b]purin- | |
| | 4-one | |
| SDZ ISQ 844 | 6,7-dimethoxy-1-(3,4-dimethoxyphenyl)-3- | 3, 4 |
| | hydroxymethyl-3,4-dihydroisoquinoline | |
| SDZ MKS 492 | R(+)-(8-[(1-(3,4-dimethoxyphenyl)-2- | 3 |
| | hydroxyethyl)amino]-3,7-dihydro-7-(2- | |
| | methoxyethyl)-1,3-dimethyl-1H-purine-2,6- | |
| | dione | |
| Senazodan | | 3 |
| Siguazodan | N-cyano-N′-methyl-N″-[4-(1,4,5,6-tetrahydro- | 3, 4 |
| | 4-methyl-6-oxo-3-pyridazinyl)phenyl]guanidine | |
| Sildenafil | 5-[2-ethoxy-5-(4-methyl-1- | 5 |
| | piperazinylsulfonyl)phenyl]-1-methyl-3-n- | |
| | propyl-1,6-dihydro-7H-pyrazolo[4,3- | |
| | d]pyrimidin-7-one (U.S. Pat. No. 5,250,534) | |
| SK 3530 | | 5 |
| SKF 94120 | 5-(4-acetamidophenyl)pyrazin-2(1H)-one | 3 |
| SKF 95654 | ±-5-methyl-6-[4-(4-oxo-1,4-dihydropyridin-1- | 3 |
| | yl)phenyl]-4,5-dihydro-3(2H)-pyridazinone | |
| SKF 96231 | 2-(2-propoxyphenyl)-6-purinone | 3, 4, 5 |
| SLX 2101 | | 5 |
| Sulmazole | U.S. Pat. No. 3,985,891 | 3 |
| T 0156 | 2-(2-methylpyridin-4-yl)methyl-4-(3,4,5- | 5 |
| | trimethoxyphenyl)-8-(pyrimidin-2-yl)methoxy- | |
| | 1,2-dihydro-1-oxo-2,7-naphthyridine-3- | |
| | carboxylic acid methyl ester hydrochloride | |
| T 1032 | methyl-2-(4-aminophenyl)-1,2-dihydro-1-oxo-7- | 5 |
| | (2-pyridylmethoxy)-4-(3,4,5-trimethoxyphenyl)- | |
| | 3-isoquinoline carboxylate sulfate | |
| T 440 | 6,7-diethoxy-1-[1-(2-methoxyethyl)-2-oxo-1,2- | 4 |
| | dihydropyridin-4-yl]naphthalene-2,3-dimethanol | |
| Tadalafil | (6R,12aR)-6-(1,3-benzodioxol-5-yl)-2-methyl- | 4, 5 |
| | 2,3,6,7,12,12a- | |
| | hexahydropyrazino[1,2,1,6]pyrido[3,4-b]indole- | |
| | 1,4-dione | |
| Tetomilast | 6-(2-(3,4-diethoxyphenyl)-4-thiazolyl)-2- | 4 |
| | pyridinecarboxylic acid | |
| Theophylline | 3,7-dihydro-1,3-dimethyl-1H-purine-2,6-dione | Not |
| | | selective |
| Tibenelast | 5,6-diethoxybenzo(B)thiophene-2-carboxylic | 4 |
| | acid | |
| Toborinone | (+/−)-6-[3-(3,4-dimethoxybenzylamino)-2- | 3 |
| | hydroxypropoxy]-2(1H)-quinolinone | |
| Tofimilast | 9-cyclopenty1-7-ethyl-6,9-dihydro-3-(2-thienyl)- | 4 |
| | 5H-pyrazolo(3,4-c)-1,2,4-triazolo(4,3-a)pyridine | |
| Tolafentrine | N-[4-[(4aS,10bR)-8,9-dimethoxy-2-methyl- | 3 (3B), 4 |
| | 3,4,4a,10b-tetrahydro-1H-pyrido[4,3- | (4B, 4D) |
| | c]isoquinolin-6-yl]phenyl]-4- | |
| | methylbenzenesulfonamide | |
| Torbafylline | 7-(ethoxymethyl)-3,7-dihydro-1-(5-hydroxy-5- | 4 |
| | methylhexyl)-3-methyl-1-H-purine-2,6-dione | |
| Trequinsin | 2,3,6,7-tetrahydro-9,10-dimethoxy-3-methyl-2- | 2, 3 (3B), 4 |
| | ((2,4,6-trimethylphenyl)imino)-4H-pyrimido(6, | (4B, 4D) |
| | 1-a)isoquinolin-4-one | |
| UCB 29936 | | 4 |
| UDCG 212 | 5-methyl-6-[2-(4-oxo-1-cyclohexa-2,5- | 3 |
| | dienylidene)-1,3-dihydrobenzimidazol-5-yl]-4,5- | |
| | dihydro-2H-pyridazin-3-one | |
| Udenafil | 3-(1-methyl-7-oxo-3-propyl-4H-pyrazolo[5,4- | 5 |
| | e]pyrimidin-5-yl)-N-[2-(1-methylpyrrolidin-2- | |
| | yl)ethyl]-4-propoxybenzenesulfonamide | |
| UK 114542 | 5-[2-ethoxy-5-(morpholinylacetyl) phenyl]-1,6- | 5 |
| | dihydro-1-methyl-3-propyl-7H-pyrazolo [4,3-d]- | |
| | pyrimidin-7-one | |
| UK 343664 | 3-ethyl-5-(5-((4-ethylpiperazino)sulphonyl)-2- | 5 |
| | propoxyphenyl)-2-(2-pyridylmethyl)-6,7- | |
| | dihydro-2H-pyrazolo(4,3-d)pyrimidin-7-one | |
| UK 357903 | 1-ethyl-4-{3-[3-ethyl-6,7-dihydro-7-oxo-2-(2- | 5 |
| | pyridylmethyl)-2H-pyrazolo[4,3-d] pyrimidin-5- | |
| | yl]-2-(2-methoxyethoxy)5-pyridylsulphonyl} | |
| | piperazine | |
| UK 369003 | | 5 |
| V 11294A | 3-((3-(cyclopentyloxy)-4- | 4 |
| | methoxyphenyl)methyl)-N-ethyl-8-(1- | |
| | methylethyl)-3H-purin-6-amine | |
| | monohydrochloride | |
| Vardenafil | 2-(2-ethoxy-5-(4-ethylpiperazin-1-yl-1- | 5 |
| | sulfonyl)phenyl)-5-methyl-7-propyl-3H- | |
| | imidazo(5,1-f)(1,2,4)triazin-4-one | |
| Vesnarinone | U.S. Pat. No. 4,415,572 | 3, 5 |
| Vinpocetine | (3-alpha,16-alpha)-eburnamenine-14-carboxylic | 1, 3, 4 |
| | acid ethyl ester | |
| WAY 122331 | 1-aza-10-(3-cyclopentyloxy-4-methoxyphenyl)- | 4 |
| | 7,8-dimethyl-3-oxaspiro[4.5]dec-7-en-2-one | |
| WAY 127093B | [(3S)-3-(3-cyc1opentyloxy-4-methoxyphenyl)-2- | 4 |
| | methyl-5-oxopyrazolidinyl]-N-(3- | |
| | pyridylmethyl)carboxamide | |
| WIN 58237 | 1-cyclopentyl-3-methyl-6-(4-pyridinyl)pyrazolo | 5 |
| | (3,4-d)pyrimidin-4(5H)-one | |
| WIN 58993 | 5-methyl-6-pyridin-4-yl-3H-[1,3]thiazolo[5,4- | 3 |
| | e]□yridine-2-one | |
| WIN 62005 | 5-methyl-6-pyridin-4-yl-1,3-dihydroimidazo[4,5- | 3 |
| | e]□yridine-2-one | |
| WIN 62582 | 6-pyridin-4-yl-5-(trifluoromethyl)-1,3- | 3 |
| | dihydroimidazo[4,5-b]□yridine-2-one | |
| WIN 63291 | 6-methyl-2-oxo-5-quinolin-6-yl-1H-pyridine-3- | 3 |
| | carbonitrile | |
| WIN 65579 | 1-cyclopentyl-6-(3-ethoxy-4-pyridinyl)-3-ethyl- | 5 |
| | 1,7-dihydro-4H-pyrazolo[3,-4-d]pyrimidin-4- | |
| | one | |
| Y 20487 | 6-(3,6-dihydro-2-oxo-2H-1,3,4-thiadiazin-5-yl)- | 3 |
| | 3,4-dihydro-2(1H)-quinolinone | |
| YM 58997 | 4-(3-bromophenyl)-1,7-diethylpyrido[2,3- | 4 |
| | d]pyrimidin-2(1H)-one | |
| YM 976 | 4-(3-chlorophenyl)-1,7-diethylpyrido(2,3- | 4 |
| | d)pyrimidin-2(1H)-one | |
| Z 15370A | | 4 |
| Zaprinast | 1,4-dihydro-5-(2-propoxyphenyl)-7H-1,2,3- | 5 |
| | triazolo[4,5-d]pyrimidine-7-one | |
| Zaprinast | 2-o-propoxyphenyl-8-azapurine-6-one | 1, 5 |
| Zardaverine | 6-(4-(difluoromethoxy)-3-methoxyphenyl)- | 2, 3 (3B), 4 |
| | 3(2H)-Pyridazinone | (4B, 4D), |
| | | 7A |
| Zindotrine | 8-methyl-6-(1-piperidinyl)-1,2,4-triazolo(4,3- | |
| | b)pyridazine | |
| CR-3465 | N-[(2-quinolinyl)carbonyl]-O-(7-fluoro-2- | 3B, 4B, 4D |
| | quinolinylmethyl)-tyrosine, sodium salt | |
| HT-0712 | (3S,5S)-5-(3-Cyclopentyloxy-4-methoxy- | 4 |
| | phenyl)-3-(3-methyl-benzyl)-piperidin-2-one | |
| 4AZA-PDE4 | | 4 |
| AN-2728 | 5-(4-cyanophenoxy)-1,3-dihydro-1-hydroxy-2,1- | 4 |
| | benzoxaborole | |
| AN-2898 | 5-(3,4-dicyanophenoxy)-1-hydroxy-1,3-dihydro- | 4 |
| | 2,1-benzoxaborole | |
| AP-0679 | | 4 |
| ASP-9831 | | 4 |
| ATI-22107 | | 3 |
| Atopik | | 4 |
| AWD-12-281 | N-(3,5-dichloropyrid-4-yl)-(1-(4-fluorobenzyl)- | 4 |
| | 5-hydroxy-indole-3-yl)glyoxylic acid amide | |
| BA-41899 | 5-methyl-6-phenyl-1,3,5,6-tetrahydro-3,6- | |
| | methano-1,5-benzodiazocine-2,4-dione | |
| BAY-61-9987 | | 4 |
| BAY-65-6207 | | 11A |
| BDD-104XX | | 5, 6 |
| BIBW-22 | 4-{N-(2-Hydroxy-2- | |
| | methylpropyl)ethanolamino)-2,7-bis(cis-2,6- | |
| | dimethylmorpholino)-6-phenylpteridine | |
| | CAS Registry No. 137694-16-7 | |
| | 2-Propanol, 1-((2,7-bis(2,6-dimethyl-4- | |
| | morpholinyl)-6-phenyl-4-pteridinyl)(2- | |
| | hydroxyethyl)amino)-2-methyl-, (cis(cis))- | |
| BMS-341400 | 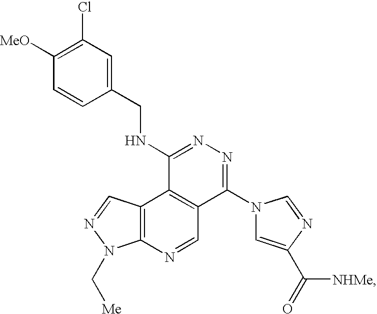 | 5 |
| CD-160130 | | 4 |
| CHF-5480 | 2-(S)-(4-lsobutyl-phenyl)-propionic acid, (Z)-2- | 4 |
| | (3,5-dichloro-pyridin-4-yl)-1-(3,4- | |
| | dimethoxy-phenyl)vinyl ester | |
| CKD-533 | | 5 |
| CT-5357 | | 4 |
| Daxalipram | (5R)-5-(4-Methoxy-3-propoxyphenyl)-5-methyl- | 4 |
| | 1,3-oxazolidin-2-one | |
| DE-103 | | 4 |
| Denbufylline | 1H-Purine-2,6-dione, 3,7-dihydro-1,3-dibutyl-7- | |
| | (2-oxopropyl)-7-Acetonyl-1,3- | |
| | dibutylxanthine | |
| DMPPO | 1,3-dimethyl-6-(2-propoxy-5- | 5 |
| | methanesulfonylamidophenyl)pyrazolo(3,4- | |
| | d)pyrimidin-4(5H)-one | |
| E-8010 | | 5 |
| ELB-526 | | 4 |
| EMD-53998 | 6-(3,6-dihydro-6-methyl-2-oxo-2H-1,3,4- | 3 |
| | thiadiazin-5-yl)-1-(3,4-dimethoxybenzoyl)- | |
| | 1,2,3,4-tetrahydro-quinoline | |
| FK-664 | 6-(3,4-Dimethoxyphenyl)-1-ethyl-4- | |
| | mesitylimino-3-methyl-3,4-dihydro-2(1H)- | |
| | pyrimidinone | |
| Flosequinan | (+−)-7-Fluoro-1-methyl-3-(methylsulfinyl)- | 3 |
| | 4(1H)-quinolinone | |
| | Manoplax | |
| | 4(1H)-Quinolinone, 7-fluoro-1-methyl-3- | |
| | (methylsulfinyl)- | |
| FR-181074 | 1-(2-chlorobenzyl)-3-isobutyryl-2-propylindole- | 5 |
| | 6-carboxamide | |
| GF-248 | 5″((propoxy),7′(4-morpholino)-phenacyl),(1- | 5 |
| | methyl-3 propyl)pyrazolo(4,3d)pyrimidin-7- | |
| | one | |
| GP-0203 | | 4 |
| HN-10200 | 2-((3-methoxy-5-methylsulfinyl)-2-thienyl)-1H- | |
| | imidazo-(4,5-c)pyridine hydrochloride | |
| KF-15232 | 4,5-dihydro-5-methyl-6-(4- | 4 |
| | ((phenylmethyl)amino)-7-quinazolinyl)- | |
| | 3(2H)-Pyridazinone | |
| KF-19514 | 5-phenyl-3-(3-pyridil)methyl-3H-imidazo(4,5- | 1, 4 |
| | c)(1,8)naphthyridin-4(5H)-one | |
| LAS-31180 | 3-methylsulfonylamino-1-methyl-4(1H)- | 3 |
| | quinolone | |
| Lificiguat | CAS Registry No. 170632-47-0 | |
| Lodenafil carbonate | bis(2-{4-[4-ethoxy-3-(1-methyl-7-oxo-3-propyl- | 5 |
| | 4,7-dihydro-1H-pyrazolo[4,3-d]pyrimidin-5- | |
| | yl)phenylsulfonyl]piperazin-1-yl}ethyl) | |
| | carbonate | |
| MEM-1917 | | 4 |
| Mepiphylline | mepyramine-theophylline-acetate | |
| Mirodenafil | 5-ethyl-2-(5-(4-(2-hydroxyethyl)piperazine-1- | |
| | sulfonyl)-2-propoxyphenyl)-7-propyl-3,5- | |
| | dihydro-4H-pyrrolo(3,2-d)pyrimidin-4-one | |
| MK-0952 | | 4 |
| NA-23063 analogs | EP0829477 | 4 |
| NCS-613 | | 4 |
| NSP-307 | | 4 |
| OPC-35564 | | 5 |
| OPC-8490 | 3,4-Dihydro-6-(4-(4-oxo-4-phenylbutyl)-1- | 3 |
| | piperazinylcarbonyl)-2(1H)-quinolinone | |
| OX-914 | | 4 |
| PDB-093 | | 5 |
| QAD-171A | | 5 |
| RPR-114597 | | 4 |
| RPR-122818 | 3(R)-(4-Methoxyphenylsulfonyl)-2(S)-methyl-7 | |
| | phenylheptanohydroxamic acid | |
| RS-25344-000 | 1-(3-nitrophenyl)-3-(4-pyridylmethyl)pyrido | 4 |
| | [2,3-d]pyrimidin-2,4(1H,3H)-dione | |
| RWJ-387273 | R290629 | 5 |
| Sophoflavescenol | 3,7-Dihydroxy-2-(4-hydroxyphenyl)-5-methoxy- | 5 |
| | 8-(3-methyl-2-butenyl)-4H-1-benzopyran-4- | |
| | one | |
| SR-265579 | 1-cyclopentyl-3-ethyl-6-(3-ethoxypyrid-4-yl)- | 5 |
| | 1H-pyrazolo[3,4-d]pyrimidin-4-one | |
| Tipelukast | 4-[6-Acetyl-3-[3-[(4-acetyl-3-hydroxy-2- | |
| | propylphenyl)sulfanyl]propoxy]-2- | |
| | propylphenoxy]butanoic acid | |
| TPI-PD3 | TPI-1100 | 4, 7 |
| UCB-101333-3 | Bioorganic & Medicinal Chemistry Letters, 16: | 4 |
| | 1834-1839 (2006) | |
| UCB-11056 | 2-(4-morpholino-6-propyl-1,3,5-triazin-2- | |
| | yl)aminoethanol | |
| UK-114502 | | 5 |
| UK-357903 | 1-ethyl-4-{3-[3-ethyl-6,7-dihydro-7-oxo-2-(2- | 5 |
| | pyridylmethyl)-2H-pyrazolo[4,3-d] pyrimidin- | |
| | 5-yl]-2-(2-methoxyethoxy)5- | |
| | pyridylsulphonyl} piperazine | |
| UK-83405 | | 4 |
| WAY-126120 | | 4 |
| WIN-61691 | Bioorganic and Medicinal Chemistry Letters, 7: | 1 |
| | 89-94(1997) | |
| XT-044 | 1-n-butyl-3-n-propylxanthine | 3 |
| XT-611 | 3,4-dipropyl-4,5,7,8-tetrahydro-3H-imidazo(1,2- | |
| | i)purin-5-one | |
| YM-393059 | N-(4,6-dimethylpyrimidin-2-yl)-4-(2-(4- | 4, 7A |
| | methoxy-3-methylphenyl)-5-(4- | |
| | methylpiperazin-1-yl)-4,5,6,7-tetrahydro-1H- | |
| | indol-1-yl)benzenesulfonamide difumarate | |
| Zoraxel | RX-10100 IR | |
| CR-3465 | N-[(2-quinolinyl)carbonyl]-O-(7-fluoro-2- | |
| | quinolinylmethyl)-L-Tyrosine, sodium salt | |
| LASSBio-294 | (2′-thienylidene)-3,4-methylenedioxy | |
| | benzoylhydrazine | |
| Serdaxin | RX-10100 XR | |
| CP 77059 | methyl 3-[2,4-dioxo-3-benzyl-1,3- | 4 |
| | dihydropyridino [2,3-d] pyrimidinyl] benzoate | |
| MX 2120 | 7-(2,2 dimethyl)propyl-1-methylxanthine | |
| UK 66838 | 6-(4-acetyl-2-methylimidazol-1-yl)-8-methyl- | |
| | 2(1H)-quinolinone | |
| CC 11050 | | 4 |
| CT 1579 | | 4 |
| Trombodipine | CAS Registry No. 113658-85-8 | |
| A 906119 | CAS Registry No. 134072-58-5 | |
| 256066 (GSK) | | 4 |

Additional PDE inhibitors are shown in Table 4.

TABLE 4

| | | |
|---|---|---|
| 5E3623 | CP 166907 | MKS 213492 |
| A 021311 | CT 1786 | N 3601 |
| ARX-111 | GRC-3566 | ND-1510 |
| ATB-901 | GRC-3590 | NR-111 |
| BFGP 385 | GRC-3785 | ORG 20494 |
| BY 244 | GRC-4039 | R-1627 |
| CH-2874 | HFV 1017 | REN 1053 |
| CH-3442 | IPL 423088 | RP 116474 |
| CH-3697 | IWF 12214 | RPR-117658 |
| CH-4139 | K 123 | SDZ-PDI-747 |
| CH-422 | KF 31334 | SKF-107806 |
| CH-673 | LAS-30989 | Vasotrope |
| CH-928 | LAS-31396 | CT 2820 |

Other PDE 1 inhibitors are described in U.S. Patent Application Nos. 20040259792 and 20050075795, incorporated herein by reference. Other PDE 2 inhibitors are described in U.S. Patent Application No. 20030176316, incorporated herein by reference. Other PDE 3 inhibitors are described in the following patents and patent applications: EP 0 653 426, EP 0 294 647, EP 0 357 788, EP 0 220 044, EP 0 326 307, EP 0 207 500, EP 0 406 958, EP 0 150 937, EP 0 075 463, EP 0 272 914, and EP 0 112 987, U.S. Pat. Nos. 4,963,561; 5,141,931, 6,897,229, and 6,156,753; U.S. Patent Application Nos. 20030158133, 20040097593, 20060030611, and 20060025463; WO 96/15117; DE 2825048; DE 2727481; DE 2847621; DE 3044568; DE 2837161; and DE 3021792, each of which is incorporated herein by reference. Other PDE 4 inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Nos. 20030187052, 20030187257, 20030144300, 20030130254, 20030186974, 20030220352, 20030134876, 20040048903, 20040023945, 20040044036, 20040106641, 20040097593, 20040242643, 20040192701, 20040224971, 20040220183, 20040180900, 20040171798, 20040167199, 20040146561, 20040152754, 20040229918, 20050192336, 20050267196, 20050049258, 20060014782, 20060004003, 20060019932, 20050267196, 20050222207, 20050222207, 20060009481; International Publication No. WO 92/079778; and Molnar-Kimber, K. L. et al. J. Immunol., 150:295 A (1993), each of which is incorporated herein by reference. Other PDE 5 inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Nos. 20030144296, 20030171384, 20040029891, 20040038996, 20040186046, 20040259792, 20040087561, 20050054660, 20050042177, 20050245544, 20060009481, each of which is incorporated herein by reference. Other PDE 6 inhibitors that can be used in the methods, compositions, and kits of the invention include those described in U.S. Patent Application Nos. 20040259792, 20040248957, 20040242673, and 20040259880, each of which is incorporated herein by reference. Other PDE 7 inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Nos. 20030186988, 20030162802, 20030191167, 20040214843, and 20060009481; International Publication WO 00/68230; and Martinez et al., J. Med. Chem. 43:683-689 (2000), Pitts et al. Bioorganic and Medicinal Chemistry Letters 14: 2955-2958 (2004), and Hunt Trends in Medicinal Chemistry 2000:November 30(2), each of which is incorporated herein by reference. Other PDE inhibitors that can be used in the methods, compositions, and kits of the invention are described in U.S. Pat. No. 6,953,774.

In certain embodiments, more than one PDE inhibitor may be employed in the invention so that the combination has activity against at least two of PDE 2, 3, 4, and 7. In other embodiments, a single PDE inhibitor having activity against at least two of PDE 2, 3, 4, and 7 is employed.

### Combinations

The invention includes the individual combination of each A2A receptor agonist with each PDE inhibitor provided herein, as if each combination were explicitly stated. In a particular example, the A2A receptor agonist is IB-MECA or chloro-IB-MECA, and the PDE inhibitor is any one or more of the PDE inhibitors described herein. In another example, the PDE inhibitor is trequinsin, zardaverine, roflumilast, rolipram, cilostazol, milrinone, papaverine, BAY 60-7550, or BRL-50481, and the A2A agonist is any one or more of the A2A agonists provided herein.

B-cell Proliferative Disorders B-cell proliferative disorders include B-cell cancers and autoimmune lymphoproliferative disease. Exemplary B-cell cancers that are treated according to the methods of the invention include B-cell CLL, B-cell prolymphocyte leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, indolent myeloma, smoldering myeloma, monoclonal gammopathy of unknown significance (MGUS), B-cell non-Hodgkin's lymphoma, small lymphocytic lymphoma, monoclonal immunoglobin deposition diseases, heavy chain diseases, mediastinal (thymic) large B-cell lymphoma, intravascular large B-cell lymphoma, primary effusion lymphoma, lymphomatoid granulomatosis, precursor B-lymphoblastic leukemia/lymphoma, Hodgkin's lymphoma (e.g., nodular lymphocyte predominant Hodgkin's lymphoma, classical Hodgkin's lymphoma, nodular sclerosis Hodgkin's lymphoma, mixed cellularity Hodgkin's lymphoma, lymphocyte-rich classical Hodgkin's lymphoma, and lymphocyte depleted Hodgkin's lymphoma), post-transplant lymphoproliferative disorder, and Waldenstrom's macroglobulineamia. A preferred B-cell cancer is multiple myeloma. Other such disorders are known in the art.

### Additional Compounds

A combination of an A2A receptor agonist and a PDE inhibitor may also be employed with an antiproliferative compound for the treatment of a B-cell proliferative disorder. Additional compounds that are useful in such methods include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors (for example, NPI-0052), CD40 inhibitors, anti-CSI antibodies, FGFR3 inhibitors, VEGF inhibitors, MEK inhibitors, cyclin D1 inhibitors, NF-1B inhibitors, anthracyclines, histone deacetylases, kinesin inhibitors, phosphatase inhibitors, COX2 inhibitors, mTOR inhibitors, calcineurin antagonists, IMiDs, or other agents used to treat proliferative diseases. Specific examples are shown in Tables 5 and 6.

TABLE 5

| | | |
|---|---|---|
| 17-AAG (KOS-953) | 1D09C3 | Activated T cells |
| AE 941 | Aflibercept | AG 490 |
| Alemtuzumab | Alitretinoin oral - Ligand | Alvocidib |
| | Pharmaceuticals | |
| AMG162 (denosumab, | Anti-CD38 antibodies | Anti-CD38 monoclonal |
| osteoprotegerin, OPG) | | antibody AT13/5 |
| Anti-CD46 human | Anti-CD5 monoclonal | Anti-HM1-24 monoclonal |
| monoclonal antibodies | antibodies | antibody |
| Anti-MUC1 monoclonal | Antineoplaston A10 - | Antineoplaston AS2 1 - |
| antibody - United | injection | injection |
| Therapeutics/ViRexx | | |
| Medical Corp | | |
| AP23573 | APC 8020 | Aplidin ® |
| Apo2L/TRAIL | Apomine ™ (SR-45023A) | AR20.5 |
| Arsenic trioxide | AT 101 | Atacicept (TACI-Ig) |
| Atiprimod | Atiprimod | ATN 224 |
| Avastin ™ (bevacizumab, | AVN944 | Azathioprine |
| rhuMAb-VEGF) | | |
| B-B4-DMI | BCX-1777 (forodesine) | Belinostat |
| Bendamustine (SDX-105) | Benzylguanine | Beta alethine |
| Bexxar (Iodine I 131 | BIBF-1120 | Bortezomib (VELCADE ®) |
| tositumomab) | | |
| Breva-Rex ® | Brostallicin | Bufexamac |
| BX 471 | Cadi-05 | Cancer immunotherapies - |
| | | Cell Genesys |
| Carmustine | CC 4047 | CC007 |
| CC11006 | CCI-779 | CD74-targeted therapeutics |
| Celebrex (celecoxib) | CERA (Continuous | CHIR-12.12 |
| | Erythropoiesis Receptor | |
| | Activator) | |
| cKap | Clodronic acid | CNTO 328 |
| CP 751871 | CRB 15 | Curcumin |
| Cyclophosphamide | Danton | Darinaparsin |
| Dasatinib | Daunorubicin liposomal | Defibrotide |
| Dexamethasone | Dexniguldipine | DHMEQ |
| Dimethylcelecoxib | DOM1112 | Doxorubicin |
| Doxorubicin liposomal | Doxycycline | Elsilimomab |
| (PNU-108112) - ALZA | | |
| EM164 | ENMD 0995 | Erbitux, cetuximab |
| Ethyol ® (amifostine) | Etoposide | Fibroblast growth factor |
| | | receptor inhibitors |
| Fludarabine | Fluphenazine | FR901228 (depsipeptide) |
| G3139 | Gallium Maltolate | GCS 100 |
| GCS-100 | GCS-100LE | GRN 163L |
| GVAX ® Myeloma Vaccine | GW654652 | GX15-070 |
| HGS-ETR1 (TRM-1, | Highly purified | Histamine dihydrochloride |
| mapatumumab) | hematopoietic stem cells | injection - EpiCept |
| | | Corporation |
| hLL1 | Holmium-166 DOTMP | HSV thymidine kinase gene |
| | | therapy |
| HuLuc63 | HuMax-CD38 | huN901-DM1 |
| Idarubicin | Imexon - Heidelberg | Imexon (plimexon) - |
| | Pharma | AmpliMed |
| IMMU 110 | Incadronic acid | Interferon-alpha-2b |
| IPI 504 | Irinotecan | ISIS 345794 |
| Isotretinoin | ITF 2357 | Kineret ™ (anakinra) |
| KOS-1022 (alvespimycin | KRX-0401, perifosine | LAF 389 |
| HCl; 17-DMAG; | | |
| NSC707545) | | |
| LBH589 | Lenalidomide (Revlimid ®) | Lestaurtinib |
| LPAAT-β inhibitors | Lucatumumab | LY2181308 |
| Melphalan | Menogaril | Midostaurin |
| Minodronic acid | MK 0646 | MOR202 |
| MS-275 | Multiple myeloma vaccine - | MV-NIS |
| | GTC | |
| Myeloma vaccine - Onyvax | MyelomaCide | Mylovenge |
| Nexavar ® (BAY 43-9006, | Noscapine | NPI 0052 |
| sorafenib, sorafenib | | |
| tosylate) | | |
| O-6-benzyl-guanine | Obatoclax | Oblimersen |
| OGX-427 | Paclitaxel | Pamidronic acid |
| Panzem ™ (2-meth- | Parthenolide | PD173074 |
| oxyestradiol, 2ME2) | | |
| Phosphostim | PI 88 | Plitidepsin |
| PR-171 | Prednisone | Proleukin ® (IL-2, |
| | | Interleukin-2) |
| PX-12 | PXD101 | Pyroxamide |
| Quadrarnet ® (EDTMP, | RAD001 (everolimus) | Radiolabelled BLyS |
| samarium-153 ethylene | | |
| diamine tetramethylene | | |
| phosphonate Samarium) | | |
| RANK-Fc | Rituximab | Romidepsin |
| RTA402 | Samarium 153 SM | Sant 7 |
| | lexidronam | |
| SCIO-469 | SD-208 | SDX-101 |
| Seleciclib | SF1126 | SGN 40 |
| SGN-70 | Sirolimus | Sodium Stibogluconate |
| | | (VQD-001) |
| Spironolactone | SR 31747 | SU5416 |
| SU6668 | Tanespimycin | Temodar ® (temozolomide) |
| Thalidomide | Thrombospondin-1 | Tiazofurine |
| Tipifarnib | TKI 258 | Tocilizumab (atlizumab) |
| Topotecan | Tretinoin | Valspodar |
| Vandetanib (Zactima ™) | Vatalanib | VEGF Trap (NSC 724770) |
| Vincristine | Vinorelbine | VNP 4010M |
| Vorinostat | Xcytrin (motexafin | XL999 |
| | gadolinium) | |
| ZIO-101 | Zoledronic acid | ZRx 101 |
| 1D09C3 | detumomab | IdioVax |
| A-623 | diazeniumdiolates | IL-1 receptor Type 2 |
| AEW-541 | DOM-1112 | Il-12 |
| agatolimod | dovitinib | IL-6 trap |
| Alfaferone | doxil (pegylated dox) | ImMucin |
| anti CD22/N97A | doxorubicin-LL2 conjugate | INCB-18424 |
| anti-CD20-IL2 | elsilimomab | infliximab |
| immunocytokine | | |
| anti-CD46 mAb | enzastaurin | IPH-1101 |
| APO-010 | farnesyl transferase | IPH-2101 |
| | inhibitors | |
| apolizumab | fostamatinib disodium | ISF-154 |
| AR-726 | gadolinium texaphyrin | JAK tyrosine kinase |
| | | inhibitors |
| B-B4-DC1 | GRN-163L | K562/GM-CSF |
| B-B4-DM1 | GVAX | KRX-0402 |
| bectumomab | HuMax-CD38 | L1R3 |
| BHQ-880 | Oncolym | LMB-2 |
| blinatumomab | Onyvax-M | lomustine |
| BT-062 | P-276-00 | LY-2127399 |
| carfilzomib | pazopanib | LymphoRad-131 |
| CAT-3888 | PD-332991 | mAb-1.5.3 |
| CAT-8015 | perifosine | mapatumumab |
| CB-001 | PG-120 | masitinib |
| CC-394 | phorboxazole A, Hughes | MDX-1097 |
| | Institute | |
| CEP-18770 | pomalidomide | XL-228 |
| clofarabine | ProMabin | XmAb-5592 |
| CT-32228 | MGCD-0103 | YM-155 |
| cyclolignan | milatuzumab | talmapimod |
| picropodophyllin | | |
| CYT-997 | mitumprotimut-t | tamibarotene |
| dacetuzumab | MM-014 | temsirolimus |
| dasatinib | MOR-202 | TG-1042 |
| DaunoXome | MyelomaScan | Vitalethine |
| denosumab | N,N-disubstituted alanine | SF-1126 |
| PS-031291 | ofatumumab | SNS-032 |
| PSK-3668 | SAR-3419 | SR-45023A |
| R-7159 | SCIO-323 | STAT-3 inhibitors |
| Rebif | SDX-101 | XBP-1 peptides |
| retaspimycin | SDZ-GLI-328 | Xcellerated T cells |
| Reviroc | seliciclib | semaxanib |
| Roferon-A | | |

Combinations of the invention may also be employed with combinations of antiproliferative compounds. Such additional combinations include CHOP (cyclophosphamide, vincristine, doxorubicin, and prednisone), VAD (vincristine, doxorubicin, and dexamethasone), MP (melphalan and prednisone), DT (dexamethasone and thalidomide), DM (dexamethasone and melphalan), DR (dexamethasone and Revlimid), DV (dexamethasone and Velcade), RV (Revlimid and Velcade), and cyclophosphamide and etoposide.

Additional compounds related to bortezomib that may be used in the invention are described in U.S. Pat. Nos. 5,780,454, 6,083,903, 6,297,217, 6,617,317, 6,713,446, 6,958,319, and 7,119,080. Other analogs and formulations of bortezomib are described in U.S. Pat. Nos. 6,221,888, 6,462,019, 6,472,158, 6,492,333, 6,649,593, 6,656,904, 6,699,835, 6,740,674, 6,747,150, 6,831,057, 6,838,252, 6,838,436, 6,884,769, 6,902,721, 6,919,382, 6,919,382, 6,933,290, 6,958,220, 7,026,296, 7,109,323, 7,112,572, 7,112,588, 7,175,994, 7,223,554, 7,223,745, 7,259,138, 7,265,118, 7,276,371, 7,282,484, and 7,371,729.

Additional compounds related to lenalidomide that may be used in the invention are described in U.S. Pat. Nos. 5,635,517, 6,045,501, 6,281,230, 6,315,720, 6,555,554, 6,561,976, 6,561,977, 6,755,784, 6,908,432, 7,119,106, and 7,189,740. Other analogs and formulations of lenalidomide are described in U.S. Pat. Nos. RE40,360, 5,712,291, 5,874,448, 6,235,756, 6,281,230, 6,315,720, 6,316,471, 6,335,349, 6,380,239, 6,395,754, 6,458,810, 6,476,052, 6,555,554, 6,561,976, 6,561,977, 6,588,548, 6,755,784, 6,767,326, 6,869,399, 6,871,783, 6,908,432, 6,977,268, 7,041,680, 7,081,464, 7,091,353, 7,115,277, 7,117,158, 7,119,106, 7,141,018, 7,153,867, 7,182,953, 7,189,740, 7,320,991, 7,323,479, and 7,329,761.

Further compounds that may be employed with the combinations of the invention are shown in Table 6.

TABLE 6

| | | |
|---|---|---|
| 6-Mercaptopurine | Gallium (III) Nitrate | Altretamine |
| | Hydrate | |
| Anastrozole | Bicalutamide | Bleomycin |
| Busulfan | Camptothecin | Capecitabine |
| Carboplatin | Chlorambucil | Cisplatin |
| Cladribine | Cytarabine | Dacarbazine |
| Dactinomycin | Docetaxel | Epirubicin Hydrochloride |
| Estramustine | Exemestane | Floxuridine |
| Fluorouracil | Flutamide | Fulvestrant |
| Gemcitabine | Hydroxyurea | Ifosfamide |
| Hydrochloride | | |
| Imatinib | Iressa | Ketoconazole |
| Letrozole | Leuprolide | Levamisole |
| Lomustine | Mechlorethamine | Megestrol acetate |
| | Hydrochloride | |
| Methotrexate | Mitomycin | Mitoxantrone |
| | | Hydrochloride |
| Nilutamide | Oxaliplatin | Pemetrexed |
| Plicamycin | Prednisolone | Procarbazine |
| Raltitrexed | Rofecoxib | Streptozocin |
| Suramin | Tamoxifen Citrate | Teniposide |
| Testolactone | Thioguanine | Thiotepa |
| Toremifene | Vinblastine Sulfate | Vindesine |

A combination of an A2A receptor agonist and a PDE inhibitor may also be employed with IL-6 for the treatment of a B-cell proliferative disorder. If not by direct administration of IL-6, patients may be treated with agent(s) to increase the expression or activity of IL-6. Such agents may include other cytokines (e.g., IL-1 or TNF), soluble IL-6 receptor α (sIL-6R α), platelet-derived growth factor, prostaglandin E1, forskolin, cholera toxin, dibutyryl cAMP, or IL-6 receptor agonists, e.g., the agonist antibody MT-18, K-7/D-6, and compounds disclosed in U.S. Pat. Nos. 5,914,106, 5,506,107, and 5,891,998.

### Administration

In particular embodiments of any of the methods of the invention, the compounds are administered within 28 days of each other, within 14 days of each other, within 10 days of each other, within five days of each other, within twenty-four hours of each other, or simultaneously. The compounds may be formulated together as a single composition, or may be formulated and administered separately. Each compound may be administered in a low dosage or in a high dosage, each of which is defined herein.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. In one example, RPL554 is administered intranasally.

In combination therapy, the dosage and frequency of administration of each component of the combination can be controlled independently. For example, one compound may be administered three times per day, while a second compound may be administered once per day. Combination therapy may be given in on-and-off cycles that include rest periods so that the patient's body has a chance to recover from any as yet unforeseen side effects. The compounds may also be formulated together such that one administration delivers both compounds.

### Formulation of Pharmaceutical Compositions

The administration of a combination of the invention may be by any suitable means that results in suppression of proliferation at the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 21st edition, 2005, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, all agents may be formulated together or separately. Desirably, all agents are formulated together for the simultaneous or near simultaneous administration of the agents. Such co-formulated compositions can include the A2A receptor agonist and the PDE inhibitor formulated together in the same pill, capsule, liquid, etc. It is to be understood that, when referring to the formulation of “A2A agonist/PDE inhibitor combinations,” the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Dosages

Generally, the dosage of the A2A receptor agonist is 0.1 mg to 500 mg per day, e.g., about 50 mg per day, about 5 mg per day, or desirably about 1 mg per day. The dosage of the PDE inhibitor is, for example, 0.1 to 2000 mg, e.g., about 200 mg per day, about 20 mg per day, or desirably about 4 mg per day.

Dosages of antiproliferative compounds are known in the art and can be determined using standard medical techniques.

Administration of each drug in the combination can, independently, be one to four times daily for one day to one year.

The following examples are to illustrate the invention. They are not meant to limit the invention in any way.

### EXAMPLE 1

### Materials and Methods

### Tumor Cell Culture

The MM.1S, MM.1R, H929, MOLP-8, EJM, INA-6, ANBL6, KSM-12-PE, OPM2, and RPMI-8226 multiple myeloma cell lines, as well as the Burkitt's lymphoma cell line GA-10 and the non-Hodgkin's lymphoma cell lines Farage, SU-DHL6, and Karpas 422 were cultured at 37° C. and 5% CO₂ in RPMI-1640 media supplemented with 10% FBS. ANBL6 and INA-6 culture media was also supplemented with 10 ng/ml IL-6. The OCI-ly10 cell line was cultured using RPMI-1640 media supplemented with 20% human serum. MM.1S, MM.1R, OCI-ly10, Karpas 422, and SU-DHL6 cells were provided by the Dana Farber Cancer Institute. H929, RPMI-8226, GA-10, and Farage cells were from ATCC (Cat #'s CCL-155, CRL-9068, CRL-2392 and CRL-2630 respectively). MOLP-8, EJM, KSM-12-PE, and OPM2 were from DSMZ. The ANBL6 and INA-6 cell lines were provided by the M. D. Anderson Cancer Research Center.

### Compounds

Compounds were prepared in DMSO at 1000× the highest desired concentration. Master plates were generated consisting of serially diluted compounds in 2- or 3-fold dilutions in 384-well format. For single agent dose response curves, the master plates consisted of 9 individual compounds at 12 concentrations in 2- or 3-fold dilutions. For combination matrices, master plates consisted of individual compounds at 6 or 9 concentrations at 2- or 3-fold dilutions.

siRNA and Transcript Quantification

siRNA to adenosine receptor A1, A2A, A3, PDE 2A, PDE 3B, PDE 4B, PDE 4D and PDE 7A, and control siRNA siCON were purchased from Dharmacon. A2B siRNA was purchased from Invitrogen. Electroporations were performed using an Amaxa Nucleoporator (program S-20) and solution V. siRNAs were used at 50 nM. Electroporation efficiency (MM.1R cells) was 87% as determined using siGLO (Dharmacon), and cells remained 89% viable 24 hours post electroporation. RNA was isolated using Qiagen RNAeasy kits, and targets quantified by RT-PCR using gene specific primers purchased from Applied Biosystems.

### Anti-Proliferation Assay

Cells were added to 384-well plates 24 hours prior to compound addition such that each well contained 2000 cells in 35 μL of media. Master plates were diluted 100× (1 μL into 100 μL) into 384-well dilution plates containing only cell culture media. 4.5 μL from each dilution plate was added to each assay plate for a final dilution of 1000×. To obtain combination data, two master plates were diluted into the assay plates. Following compound addition, assay plates were kept at 37° C. and 5% CO₂ for 72 hours. Thirty microliters of ATPLite (Perkin Elmer) at room temperature was then added to each well. Final amount of ATP was quantified within 30 minutes using ATPLite luminescent read-out on an Envision 2103 Multilabel Reader (Perkin Elmer). Measurements were taken at the top of the well using a luminescence aperture and a read time of 0.1 seconds per well.

The percent inhibition (% I) for each well was calculated using the following formula:

% I=[(avg. untreated wells−treated well)/(avg. untreated wells)]×100.

The average untreated well value (avg. untreated wells) is the arithmetic mean of 40 wells from the same assay plate treated with vehicle alone. Negative inhibition values result from local variations in treated wells as compared to untreated wells.

Single agent activity was characterized by fitting a sigmoidal function of the form I═IₘₐₓC^{α}/[C^{α}+EC₅₀^{α}] with least squares minimization using a downhill simplex algorithm (C is the concentration, EC₅₀ is the agent concentration required to obtain 50% of the maximum effect, and a is the sigmoidicity). The uncertainty of each fitted parameter was estimated from the range over which the change in reduced chi-squared was less than one, or less than minimum reduced chi-squared if that minimum exceeded one, to allow for underestimated σ_{I} errors.

Single agent curve data were used to define a dilution series for each compound to be used for combination screening in a 6×6 matrix format. Using a dilution factor f of 2, 3, or 4, depending on the sigmoidicity of the single agent curve, five dose levels were chosen with the central concentration close to the fitted EC₅₀. For compounds with no detectable single agent activity, a dilution factor of 4 was used, starting from the highest achievable concentration.

The Loewe additivity model was used to quantify combination effects. Combinations were ranked initially by Additivity Excess Volume, which is defined as ADD Volume=ΣC_{X}, C_{Y} (I_{data}−I_{Loewe}). where I_{Loewe}(C_{X}, C_{Y}) is the inhibition that satisfies (C_{X}/EC_{X})+(C_{Y}/EC_{Y})=1, and EC_{X,Y} are the effective concentrations at I_{Loewe} for the single agent curves. A “Synergy Score” was also used, where the Synergy Score S=log f_{X} log f_{Y} ΣI_{data}(I_{data}−I_{Loewe}), summed over all non-single-agent concentration pairs, and where log f_{X,Y} is the natural logarithm of the dilution factors used for each single agent. This effectively calculates a volume between the measured and Loewe additive response surfaces, weighted towards high inhibition and corrected for varying dilution factors. An uncertainty σ_{S} was calculated for each synergy score, based on the measured errors for the I_{data} values and standard error propagation.

### Chronic Lymphocytic Leukemia (CLL) Isolation and Cell Culture

Blood samples were obtained in heparinized tubes with IRB-approved consent from flow cytometry-confirmed B-CLL patients that were either untreated or for whom at least 1 month had elapsed since chemotherapy. Patients with active infections or other serious medical conditions were not included in this study. Patients with white blood cell counts of less than 15,000/μl by automated analysis were excluded from this study. Whole blood was layered on Ficoll-Hystopaque (Sigma), and peripheral blood mononuclear cells (PBMC) isolated after centrification. PBMCs were washed and resuspended in complete media [RPMI-1640 (Mediatech) supplemented with 10% fetal bovine serum (Sigma), 20 mM L-glutamine, 100 IU/ml penicillin, and 100 μg/ml streptomycin (Mediatech)]. One million cells were stained with anti-CD5-PE and anti-CD19-PE-Cy5 (Becton Dickenson, Franklin Lakes N.J.). The percentage of B-CLL cells was defined as the percentage of cells doubly expressing CD5 and CDl9, as determined by flow cytometry.

### Apoptosis Assays

Approximately five million cells per well were seeded in 96-well plates (BD, Franklin Lakes N.J.) and incubated for one hour at 37° C. in 5% CO₂. Compound master plates were diluted 1:50 into complete media to create working compound dilutions. Compound crosses were then created by diluting two working dilution plates 1:10 into each plate of cells. After drug addition, cells were incubated for 48 hours at 37° C. with 5% CO₂. Hoechst 33342 (Molecular Probes, Eugene Oreg.) at a final concentration of 0.25 μg/1 mL was added to each well, and the cells incubated at 37° C. for an additional ten minutes before being placed on ice until analysis. Plates were then analyzed on a LSR-II flow cytometer (Becton Dickenson, Franklin Lakes, N.J.) equipped with the High Throughput Sampling (HTS) option in high throughput mode. The dye was excited using a 355 nm laser, and fluorescence was detected utilizing a 450/50 nm bandpass filter. The apoptotic fraction was calculated using FlowJo software (Tree Star Inc., Ashland, Oreg.) after excluding debris by a FSC/SSC gate and subsequently gating for cells that accumulate the Hoechst dye.

### EXAMPLE 2

The RPMI-8226, MM.1S, MM.1R, and H929 mM cell lines were used to examine the activity of various compounds. The synergy scores obtained are provided in the following tables.

TABLE 7
Summary of synergy scores for compounds that synergize with the
adenosine receptor agonist ADAC in one or more MM cell line
(RPMI-8226, MM.1S, MM.1R, and H929)

| | RPMI- | | | |
|---|---|---|---|---|
| | 8226 | H929 | MM.1S | MM.1R |
| Papaverine hydrochloride | 1.158 | 1.193 | 3.554 | 3.395 |
| Trequinsin hydrochloride | 0.9183 | 3.044 | 6.619 | 6.47 |
| Rolipram | 0.4277 | 1.114 | 1.147 | 4.105 |
| RO-20-1724 | 0.51 | 1.1 | 1.71 | 3.42 |
| Dipyridamole | 0.62 | 2.05 | 1.18 | 1.34 |

TABLE 8
Summary of synergy scores for compounds that synergize with the
adenosine receptor agonist HE-NECA in one or more MM cell line
(RPMI-8226, MM.1S, MM.1R, and H929)

| | RPMI- | | | |
|---|---|---|---|---|
| | 8226 | H929 | MM.1S | MM.1R |
| Papaverine hydrochloride | 0.3933 | 1.025 | 2.087 | 2.128 |
| Trequinsin hydrochloride | 0.793 | 3.141 | 7.235 | 4.329 |
| BAY 60-7550 | 0.7784 | 1.933 | 2.364 | N.D. |
| R-(−)-Rolipram | 1.16 | 2.148 | 2.965 | N.D. |
| Rolipram | 0.2845 | 1.089 | 1.076 | N.D. |
| Cilostamide | 0.2381 | 1.67 | 1.637 | 1.692 |
| Cilostazol | 0.2486 | 0.6849 | 1.849 | N.D. |
| Roflumilast | 0.466 | 0.98 | 2 | N.D. |
| Zardaverine | 0.43 | 3.39 | 4.39 | N.D. |
| BRL-50481 | 0.147 | 0.193 | 1.38 | N.D. |

### EXAMPLE 3

The RPMI-8226, MM.1S, MM.1R, and H929 mM cell lines were used to examine the activity of various compounds. The synergy scores obtained are provided in the following tables.

TABLE 9
Summary of synergy scores for compounds that synergize with the
adenosine receptor agonist CGS-21680 in one or more MM cell lines
(RPMI-8226, MM.1S, MM.1R, and H929)

| | RPMI | | | |
|---|---|---|---|---|
| | 8226 | H929 | MM.1S | MM.1R |
| Trequinsin | 0.72 | 3.33 | 6.26 | 6.57 |
| Zardaverine | 0.13 | 3.75 | 3.64 | 2.15 |
| BAY 60-7550 | 0.76 | 3.86 | 3.85 | 4.59 |
| R-(−)-Rolipram | 2.03 | 1.93 | 1.92 | 4.54 |
| Cilostazol | 0.37 | 1.12 | 4.09 | 1.57 |
| Roflumilast | 0.69 | 3.71 | 3.82 | 3.61 |
| BRL-50481 | 0.19 | 0.34 | 1.78 | 1.22 |
| Ibudilast | 0.47 | 1.76 | 2.22 | 2.29 |

TABLE 10
Summary of synergy scores for compounds that synergize with the
adenosine receptor agonist regadenoson in one or more MM cell
lines (RPMI-8226, MM.1S, MM.1R, and H929)

| | RPMI 8226 | H929 | MM.1S | MM.1R |
|---|---|---|---|---|
| Trequinsin | 0.4 | 1.99 | 1.85 | 2.8 |
| Zardaverine | 0.52 | 1.02 | 1.45 | 1.49 |
| BAY 60-7550 | 0.98 | 1.89 | 0.91 | 3.07 |
| R-(−)-Rolipram | 0.63 | 1.91 | 1.83 | 3.62 |
| Cilostazol | 0.12 | 1.34 | 1.85 | 0.76 |
| Roflumilast | 1.12 | 2.7 | 3.56 | 5.83 |
| BRL-50481 | 0.39 | 0.19 | 0.82 | 1.09 |
| Ibudilast | 0.29 | 1.08 | 0.37 | 1 |

Representative 6×6 data for compounds that have synergistic anti-proliferative activity in combination with adenosine receptor agonists are shown in Tables 11-19 below. Inhibition of proliferation was measured as described above, after incubation of cells with test compound(s) for 72 hours. The effects of various concentrations of single agents or drugs in combination were compared to control wells (MM cells not treated with drugs). The effects of agents alone and in combination are shown as percent inhibition of cell proliferation.

TABLE 11
Antiproliferative activity of HE-NECA and trequinsin against human
multiple myeloma cells (MM.1S) (Percent inhibition of ATP in MM.1S
cells)

| | |
|---|---|
| HE-NECA | Trequinsin (μM) |

| (μM) | 30.5 | 10.17 | 3.39 | 1.13 | 0.377 | 0 |
|---|---|---|---|---|---|---|
| 2.03 | 95 | 93 | 91 | 94 | 94 | 86 |
| 0.677 | 96 | 92 | 92 | 91 | 90 | 80 |
| 0.226 | 95 | 91 | 91 | 91 | 89 | 83 |
| 0.0752 | 96 | 92 | 91 | 89 | 88 | 79 |
| 0.0251 | 96 | 93 | 93 | 93 | 90 | 78 |
| 0 | 68 | 26 | 10 | 0.96 | 7.4 | 0.6 |

TABLE 12
Antiproliferative activity of ADAC and trequinsin against human
multiple myeloma cells (MM.1S) (Percent inhibition of ATP in MM.1S
cells)

| Trequinsin | ADAC (μM) | | | | | |
|---|---|---|---|---|---|---|
| Hydrochloride (μM) | 31.6 | 15.8 | 7.9 | 3.95 | 1.975 | 0 |
| 30.5 | 96 | 96 | 96 | 96 | 98 | 87 |
| 10.2 | 92 | 93 | 91 | 92 | 86 | 30 |
| 3.39 | 90 | 88 | 88 | 87 | 85 | 5.4 |
| 1.13 | 85 | 87 | 81 | 80 | 72 | 3.7 |
| 0.377 | 84 | 75 | 80 | 69 | 56 | 0.44 |
| 0 | 60 | 66 | 57 | 49 | 37 | 7.9 |

TABLE 13
Antiproliferative activity of HE-NECA and BAY 60-7550 against human
multiple myeloma cells (MM.1S) (Percent inhibition of ATP in MM.1S
cells)

| | BAY 60-7550 (μM) | | | | | |
|---|---|---|---|---|---|---|
| HE-NECA (nM) | 11.8 | 5.9 | 2.95 | 1.475 | 0.7375 | 0 |
| 20.3 | 83 | 74 | 70 | 85 | 82 | 67 |
| 6.77 | 80 | 75 | 62 | 82 | 70 | 59 |
| 2.26 | 71 | 53 | 52 | 68 | 59 | 41 |
| 0.752 | 44 | 30 | 17 | 42 | 31 | 23 |
| 0.251 | 25 | 9.9 | 9.5 | 15 | 15 | 3.4 |
| 0 | 13 | 6 | 4 | −3.6 | −9.4 | 0.27 |

TABLE 14
Antiproliferative activity of chloro-IB-MECA and papaverine against
human multiple myeloma cells (MM.1S) (Percent inhibition of ATP in
MM.1S cells)

| | Cl-IB-MECA (μM) | | | | | |
|---|---|---|---|---|---|---|
| Papaverine (μM) | 3.1 | 1.55 | 0.775 | 0.3875 | 0.19375 | 0 |
| 30.8 | 100 | 98 | 98 | 96 | 94 | 78 |
| 15.4 | 97 | 94 | 91 | 90 | 88 | 63 |
| 7.7 | 93 | 86 | 84 | 82 | 75 | 49 |
| 3.85 | 81 | 79 | 75 | 66 | 54 | 32 |
| 1.92 | 70 | 64 | 60 | 48 | 39 | 14 |
| 0 | 55 | 51 | 39 | 29 | 20 | 0.65 |

TABLE 15
Antiproliferative activity of chloro-IB-MECA and cilostamide against
human multiple myeloma cells (MM.1S) (Percent inhibition of ATP in
MM.1S cells)

| | Cl-IB-MECA (μM) | | | | | |
|---|---|---|---|---|---|---|
| Cilostamide (μM) | 1.16 | 0.58 | 0.29 | 0.145 | 0.0725 | 0 |
| 19.7 | 90 | 80 | 63 | 74 | 52 | 60 |
| 6.57 | 75 | 72 | 39 | 32 | 31 | 4.2 |
| 2.19 | 67 | 51 | 43 | 22 | 19 | 13 |
| 0.730 | 63 | 46 | 41 | 25 | 18 | −0.84 |
| 0.243 | 60 | 49 | 37 | 28 | 6.7 | 5.2 |
| 0 | 48 | 41 | 30 | 22 | 12 | 3.5 |

TABLE 16
Antiproliferative activity of chloro-IB-MECA and roflumilast against
human multiple myeloma cells (MM.1S) (Percent inhibition of ATP in
MM.1S cells)

| | Roflumilast (μM) | | | | | |
|---|---|---|---|---|---|---|
| Cl-IB-MECA (μM) | 1.01 | 0.505 | 0.252 | 0.126 | 0.0631 | 0 |
| 3.1 | 81 | 79 | 79 | 76 | 79 | 60 |
| 1.03 | 76 | 76 | 73 | 75 | 72 | 55 |
| 0.344 | 62 | 66 | 63 | 56 | 54 | 28 |
| 0.115 | 38 | 36 | 24 | 29 | 17 | 12 |
| 0.0383 | 14 | 10 | 10 | 9.5 | 6.7 | 2.1 |
| 0 | 7.5 | 11 | −3.5 | 1.5 | −7.1 | −3.1 |

TABLE 17
Antiproliferative activity of chloro-IB-MECA and zardaverine
against human multiple myeloma cells (MM.1S)
(Percent inhibition of ATP in MM.1S cells)

| | Zardaverine (μM) | | | | | |
|---|---|---|---|---|---|---|
| Cl-IB-MECA (μM) | 30.3 | 15.2 | 7.58 | 3.79 | 1.89 | 0 |
| 3.1 | 91 | 91 | 90 | 88 | 82 | 64 |
| 1.03 | 90 | 89 | 87 | 84 | 79 | 57 |
| 0.344 | 85 | 82 | 77 | 73 | 69 | 37 |
| 0.115 | 64 | 59 | 54 | 43 | 35 | 19 |
| 0.0383 | 31 | 28 | 15 | 23 | 15 | 12 |
| 0 | 14 | 5.1 | 13 | −1.8 | 0.11 | 2.9 |

TABLE 18
Antiproliferative activity of HE-NECA and RO-20-1724
Against human multiple myeloma cells (MM.1S)
(Percent inhibition of ATP in MM.1S cells)

| | RO-20-1724 (μM) | | | | | |
|---|---|---|---|---|---|---|
| HE-NECA (nM) | 36.4 | 18.2 | 9.1 | 4.55 | 2.28 | 0 |
| 20.3 | 87 | 85 | 84 | 79 | 72 | 54 |
| 6.77 | 86 | 81 | 79 | 72 | 68 | 46 |
| 2.26 | 81 | 76 | 75 | 59 | 62 | 31 |
| 0.752 | 61 | 57 | 48 | 38 | 37 | 22 |
| 0.251 | 25 | 29 | 27 | 21 | 29 | 5.4 |
| 0 | 1.4 | 10 | 7 | 11 | 2.3 | 10 |

TABLE 19
Antiproliferative activity of HE-NECA and R-(−)-Rolipram
against human multiple myeloma cells (MM.1S) (Percent
inhibition of ATP in MM.1S cells)

| | R-(−)-Rolipram (μM) | | | | | |
|---|---|---|---|---|---|---|
| HE-NECA (nM) | 6.13 | 3.06 | 1.53 | 0.766 | 0.383 | 0 |
| 20.3 | 93 | 91 | 86 | 80 | 74 | 64 |
| 6.77 | 91 | 89 | 82 | 75 | 67 | 53 |
| 2.26 | 84 | 85 | 70 | 69 | 58 | 40 |
| 0.752 | 73 | 61 | 44 | 34 | 37 | 19 |
| 0.251 | 86 | 4.9 | −2.8 | 9.9 | 4.8 | 4.5 |
| 0 | −9.8 | −5.6 | −6.3 | −8.4 | −6.1 | 1.3 |

### EXAMPLE 4

### The Cytokine IL-6 Potentiates Adenosine Receptor Agonist Cell Killing

The localization of MM cells to bone is critical for pathogenesis. In this microenvironment, the interaction of MM cells with bone marrow stromal cells stimulates the expansion of the tumor cells through the enhanced expression of chemokines and cytokines which stimulate MM cell proliferation and protect from apoptosis. Interleukin-6 (IL-6) is the best characterized growth and survival factor for MM cells. IL-6 can trigger significant MM cell growth and protection from apoptosis in vitro. For example, IL-6 will protect cells from dexamethasone-induced apoptosis, presumably by activation of PI3K signaling. The importance of IL-6 is highlighted by the observation that IL-6 knockout mice fail to develop plasma cell tumors.

The MM.1S is an IL-6 responsive cell line that has been used to examine whether compounds can overcome the protective effects of IL-6. To examine the effect of IL-6, we first cultured MM.1S cells for 72 hours with 2-fold dilutions of dexamethasone in either the presence or absence of 10 ng/ml IL-6. Consistent with what has been described in the literature, we observe that MM.1S cell growth is stimulated (data not shown) and that cells are less sensitive to dexamethasone (2.9-fold change in IC₅₀) when cultured in the presence of IL-6 (+IL-6, IC₅₀ 0.0617 μM vs. IC₅₀ 0.179 μM, no IL-6).

We have examined the antiproliferative activity of synergistic adenosine receptor agonist combinations in the absence or presence of IL-6. In each case, we find that cells exposed to IL-6 are more sensitive to the antiproliferative effects of adenosine receptor agonist (Tables 20-25). Each of the tables provides percent inhibition of ATP in MM.1S cells (compare Table 20 with 21, Table 22 with 23 and Table 24 with 25)

TABLE 20
Antiproliferative activity of HE-NECA and trequinsin
against human multiple myeloma cells (MM.1S)

| | Trequinsin | | | | | |
|---|---|---|---|---|---|---|
| HE-NECA (nM) | 30.5 | 10.2 | 3.39 | 1.13 | 0.377 | 0 |
| 20.3 | 98 | 92 | 85 | 85 | 79 | 60 |
| 6.77 | 98 | 90 | 87 | 77 | 69 | 47 |
| 2.26 | 97 | 88 | 81 | 71 | 64 | 34 |
| 0.752 | 96 | 79 | 60 | 45 | 32 | 27 |
| 0.251 | 93 | 59 | 32 | 25 | 17 | 11 |
| 0 | 85 | 23 | 8.2 | −3.2 | −0.85 | −2.3 |

TABLE 21
Antiproliferative activity of HE-NECA and trequinsin against human
multiple myeloma cells (MM.1S) treated with 10 ng/mL IL-6

| | Trequinsin (μM) | | | | | |
|---|---|---|---|---|---|---|
| HE-NECA (nM) | 30.5 | 10.2 | 3.39 | 1.13 | 0.377 | 0 |
| 20.3 | 100 | 96 | 94 | 94 | 93 | 83 |
| 6.77 | 100 | 94 | 94 | 92 | 90 | 77 |
| 2.26 | 100 | 95 | 94 | 88 | 83 | 63 |
| 0.752 | 99 | 91 | 84 | 72 | 64 | 39 |
| 0.251 | 97 | 79 | 50 | 51 | 32 | 26 |
| 0 | 95 | 26 | 8.9 | 5.1 | −1.2 | 8.4 |

TABLE 22
Antiproliferative activity of HE-NECA and papaverine
against human multiple myeloma cells (MM.1S)

Papaverine (μM)

| HE-NECA (nM) | 20.7 | 6.9 | 2.3 | 0.767 | 0.256 | 0 |
|---|---|---|---|---|---|---|
| 20.3 | 95 | 85 | 68 | 65 | 58 | 63 |
| 6.77 | 95 | 77 | 62 | 54 | 45 | 46 |
| 2.26 | 90 | 72 | 49 | 37 | 26 | 29 |
| 0.752 | 86 | 56 | 36 | 21 | 21 | 14 |
| 0.251 | 78 | 50 | 25 | 18 | 8.8 | 11 |
| 0 | 68 | 46 | 23 | 8.8 | 9.1 | 11 |

TABLE 23
Antiproliferative activity of HE-NECA and papaverine against human
multiple myeloma cells (MM.1S) treated with 10 ng/mL IL-6

| | Papaverine (μM) | | | | | |
|---|---|---|---|---|---|---|
| HE-NECA (μM) | 20.7 | 6.9 | 2.3 | 0.767 | 0.256 | 0 |
| 20.3 | 97 | 92 | 86 | 89 | 89 | 90 |
| 6.77 | 97 | 85 | 80 | 77 | 78 | 78 |
| 2.26 | 93 | 81 | 70 | 67 | 66 | 68 |
| 0.752 | 87 | 67 | 50 | 47 | 46 | 43 |
| 0.251 | 76 | 56 | 28 | 26 | 20 | 21 |
| 0 | 70 | 46 | 7.9 | −0.1 | −2.4 | −1.9 |

TABLE 24
Antiproliferative activity of ADAC and trequinsin against human
multiple myeloma cells (MM.1S)

| | ADAC (μM) | | | | | |
|---|---|---|---|---|---|---|
| Trequinsin (μM) | 31.6 | 10.5 | 3.51 | 1.17 | 0.390 | 0 |
| 30.5 | 96 | 96 | 96 | 96 | 98 | 87 |
| 10.2 | 92 | 93 | 91 | 92 | 86 | 30 |
| 3.39 | 90 | 88 | 88 | 87 | 85 | 5.4 |
| 1.13 | 85 | 87 | 81 | 80 | 72 | 3.7 |
| 0.377 | 84 | 75 | 80 | 69 | 56 | 0.44 |
| 0 | 60 | 66 | 57 | 49 | 37 | 7.9 |

TABLE 25
Antiproliferative activity of ADAC and trequinsin against human
multiple myeloma cells (MM.1S) treated with 10 ng/mL IL-6

| | ADAC (μM) | | | | | |
|---|---|---|---|---|---|---|
| Trequinsin (μM) | 31.6 | 10.5 | 3.51 | 1.17 | 0.390 | 0 |
| 30.5 | 97 | 97 | 98 | 98 | 100 | 99 |
| 10.2 | 94 | 95 | 95 | 94 | 95 | 36 |
| 3.39 | 93 | 93 | 94 | 94 | 95 | 4.5 |
| 1.13 | 93 | 94 | 93 | 93 | 93 | 4 |
| 0.377 | 95 | 93 | 93 | 92 | 88 | 7 |
| 0 | 83 | 85 | 81 | 79 | 61 | 4.9 |

### EXAMPLE 5

### Adenosine Receptor Ligand Analysis

Multiple adenosine receptor agonists including ADAC, (S)-ENBA, 2-chloro-N-6-cyclopentyladenosine, chloro-IB-MECA, IB-MECA and HE-NECA were active and synergistic in our assays when using the RPMI-8226, H929, MM.1S and MM.1R MM cell lines. That multiple members of this target class are synergistic is consistent with the target of these compounds being an adenosine receptor. As there are four members of the adenosine receptor family (A1, A2A, A2B and A3), we have used adenosine receptor antagonists to identify which receptor subtype is the target for the synergistic antiproliferative effects we have observed.

MM.1S cells were cultured for 72 hours with 2-fold dilutions of the adenosine receptor agonist chloro-IB-MECA in either the presence or absence of the A2A-selective antagonist SCH 58261 (78 nM), the A3-selective antagonist MRS 1523 (87 nM), the A1-selective antagonist DPCPX (89 nM) or the A2B-selective antagonist MRS 1574 (89 nM). The A2A antagonist SCH58261 was the most active of the antagonists, blocking chloro-IB-MECA antiproliferative activity >50% (Table 26).

TABLE 26
Percent inhibition of cell growth by chloro-IB-MECA in
the presence of adenosine receptor antagonists

| Conc. | | | | | |
|---|---|---|---|---|---|
| Chloro-IB- | no | 78 nM | 87 nM | 89 nM | 89 nM |
| MECA | antagonist | SCH58261 | MRS1523 | DPCPX | MRS1754 |
| 3.1 μM | 70 | 28 | 69 | 64 | 71 |
| 1.5 μM | 61 | 8.1 | 54 | 47 | 50 |
| 0.77 μM | 49 | 6.4 | 48 | 38 | 57 |
| 0.39 μM | 35 | 0.5 | 33 | 18 | 13 |
| 0.19 μM | 20 | 5.2 | 19 | 7.4 | 25 |

The percent inhibition of MM.1S cell growth by chloro-IB-MECA was examined when the concentration of each antagonist was increased 2-fold. Again, the A2A antagonist SCH58261 was the most active of the compounds, a 2-fold increase in concentration blocking chloro-IB-MECA antiproliferative activity >70% (Table 27).

TABLE 27
Percent inhibition of cell growth by chloro-IB-MECA in
the presence of adenosine receptor antagonists

| Conc. Cl-IB- | no | 78 nM | 150 nM | 170 nM | 174 nM | 175 nM |
|---|---|---|---|---|---|---|
| MECA | antagonist | SCH58261 | SCH58261 | MRS1523 | DPCPX | MRS1754 |
| 3.1 μM | 70 | 28 | 16 | 74 | 60 | 72 |
| 1.5 μM | 61 | 8.1 | 4.3 | 61 | 46 | 45 |
| 0.77 μM | 49 | 6.4 | −2.5 | 51 | 36 | 52 |
| 0.39 μM | 35 | 0.5 | −2 | 38 | 17 | 14 |
| 0.19 μM | 20 | 5.2 | −3.8 | 26 | 12 | 21 |

The effect of the adenosine receptor antagonists on adenosine receptor agonist (S)-ENBA was also examined. MM.1S cells were cultured for 72 hours with 3-fold dilutions of the adenosine receptor agonist (S)-ENBA in either the presence or absence of the A2A-selective antagonist SCH 58261 (78 nM), the A3-selective antagonist MRS 1523 (183 nM), the A1-selective antagonist DPCPX (178 nM) or the A2B-selective antagonist MRS 1574 (175 nM). The A2A antagonist SCH58261 was again the most active of the antagonists. The other antagonists had marginal activity at best relative to the A2A-selective antagonist SCH58261, even though they were tested at a 2-fold higher concentration than SCH58261 (Table 28).

TABLE 28
Percent inhibition of cell growth by (S)-ENBA in the
presence of adenosine receptor antagonists

| Conc | no | 78 nM | 183 nM | 178 nM | 175 nM |
|---|---|---|---|---|---|
| (s)-ENBA | antagonist | SCH58261 | MRS1523 | DPCPX | MRS1754 |
| 14 μM | 68 | 45 | 65 | 89 | 71 |
| 4.7 μM | 52 | 12 | 52 | 77 | 47 |
| 1.6 μM | 41 | 14 | 36 | 37 | 50 |
| 0.52 μM | 19 | 6 | 14 | 18 | 10 |
| 0.17 μM | 6 | 4.5 | 10 | 2.4 | 9.3 |

The effects of the four antagonists, when adenosine receptor agonist chloro-IB-MECA is crossed with the phosphodiesterase inhibitor trequinsin are shown below. The A2A receptor antagonist SCH58261 is the most active compound. The effects of the four antagonists on synergy, when adenosine receptor agonist (S)-ENBA is crossed with the phosphodiesterase inhibitor trequinsin, are also shown below. Again, the A2A receptor antagonist SCH58261 is the most active compound. Percent inhibition of ATP in MM.1S cells is provided in each table (Tables 29-33).

TABLE 29
Antiproliferative activity of chloro-IB-MECA and trequinsin against
human multiple myeloma cells (MM.1S) after addition of 175 nM
adenosine receptor antagonist MRS 1754

| | Cl-IB-MECA (μM) | | | | | |
|---|---|---|---|---|---|---|
| Trequinsin (μM) | 2.96 | 1.48 | 0.74 | 0.37 | 0.185 | 0 |
| 29.2 | 95 | 94 | 91 | 90 | 83 | 66 |
| 9.73 | 93 | 90 | 88 | 73 | 63 | 15 |
| 3.24 | 89 | 87 | 78 | 58 | 41 | 12 |
| 1.08 | 85 | 76 | 75 | 47 | 21 | −3.1 |
| 0.360 | 81 | 73 | 53 | 46 | 6.1 | 10 |
| 0 | 72 | 45 | 51 | 14 | 21 | −13 |

TABLE 30
Antiproliferative activity of chloro-IB-MECA and trequinsin
against human multiple myeloma cells (MM.1S) after addition
of 153 nM adenosine receptor antagonist SCH58261

| | Cl-IB-MECA (μM) | | | | | |
|---|---|---|---|---|---|---|
| Trequinsin (μM) | 2.96 | 1.48 | 0.74 | 0.37 | 0.185 | 0 |
| 29.2 | 91 | 88 | 77 | 79 | 64 | 66 |
| 9.73 | 80 | 50 | 44 | 28 | 28 | 23 |
| 3.24 | 55 | 43 | 17 | 12 | 12 | 13 |
| 1.08 | 46 | 19 | 11 | 3.5 | 1.7 | −6.6 |
| 0.360 | 36 | 14 | 5.7 | 6.4 | 2.7 | 3.9 |
| 0 | 15 | 4.3 | −2.5 | −0.16 | −3.8 | 6.5 |

TABLE 31
Antiproliferative activity of chloro-IB-MECA and trequinsin
against human multiple myeloma cells (MM.1S) after addition
of 170 nM adenosine receptor antagonist MRS 1523

| | Cl-IB-MECA (μM) | | | | | |
|---|---|---|---|---|---|---|
| Trequinsin (μM) | 2.96 | 1.48 | 0.74 | 0.37 | 0.185 | 0 |
| 29.2 | 94 | 95 | 93 | 92 | 89 | 66 |
| 9.73 | 93 | 93 | 92 | 90 | 84 | 23 |
| 3.24 | 93 | 92 | 91 | 86 | 70 | 13 |
| 1.08 | 91 | 89 | 87 | 76 | 59 | −4.8 |
| 0.360 | 88 | 99 | 77 | 70 | 36 | −8.3 |
| 0 | 75 | 61 | 51 | 38 | 27 | −12 |

TABLE 32
Antiproliferative activity of chloro-IB-MECA and trequinsin against
human multiple myeloma cells (MM.1S) after addition of 174 nM
adenosine receptor aantagonist DPCPX

| | CI-IB-MECA (μM) | | | | | |
|---|---|---|---|---|---|---|
| Trequinsin (μM) | 2.96 | 1.48 | 0.74 | 0.37 | 0.185 | 0 |
| 29.2 | 94 | 94 | 93 | 90 | 82 | 64 |
| 9.73 | 94 | 92 | 89 | 77 | 60 | 22 |
| 3.24 | 91 | 91 | 81 | 64 | 30 | 7.9 |
| 1.08 | 89 | 84 | 75 | 51 | 27 | 6.6 |
| 0.360 | 84 | 76 | 61 | 32 | 14 | −0.5 |
| 0 | 60 | 46 | 36 | 17 | 12 | −7.5 |

TABLE 33
Antiproliferative activity of chloro-IB-MECA and trequinsin against
human multiple myeloma cells (MM.1S), no adenosine receptor
antagonist added

| | CI-IB-MECA (μM) | | | | | |
|---|---|---|---|---|---|---|
| Trequinsin (μM) | 2.96 | 1.48 | 0.74 | 0.37 | 0.185 | 0 |
| 29.2 | 94 | 94 | 93 | 93 | 93 | 66 |
| 9.73 | 93 | 93 | 94 | 91 | 86 | 22 |
| 3.24 | 92 | 93 | 91 | 87 | 77 | 13 |
| 1.08 | 90 | 88 | 85 | 80 | 63 | −4 |
| 0.360 | 87 | 86 | 77 | 71 | 46 | −3.6 |
| 0 | 71 | 61 | 51 | 35 | 23 | −5.1 |

The use of adenosine receptor antagonists points to the A2A receptor subtype as important for the antiproliferative effect of agonists on cell growth. We note that our results do not exclude the importance of other adenosine receptor subtypes for maximal activity.

We also examined the antiproliferative activity of adenosine receptor agonists when the MM cell line MM.1R was transfected with siRNA targeting the A1, A2A, A2B or A3 receptor. Specific gene silencing (A1, A2A, A2B, or A3) was greater than 50% as determined by real time PCR analysis 48 hours post-transfection. At 48 hours post-transfection, cells were exposed to adenosine receptor agonist, incubated an additional 72 hours, and compounds assayed for antiproliferative activity. Representative data is in Table 34. Cells transfected with adenosine receptor siRNA or a control siRNA (scrambled sequences designed so that cellular transcriptsare not targeted) were treated with the adenosine receptor agonist ADAC. While siRNA to the A1, A2B, or A3 receptor did not affect ADAC activity, an siRNA that targeted the A2A receptor reduced the adenosine receptor agonist's anitproliferative activity. Similar results were obtained with a second siRNA with specificity for different region of the A2A receptor mRNA, confirming that the reduction in adenosine receptor agonist activity is the result of specific siRNA targeting of the A2A receptor (data not shown).

TABLE 34
Antiproliferative activity of adenosine receptor agonist ADAC against
human multiple myeloma cells (MM.1R) after transfection of siRNA
silencing the adenosine receptor subtypes

| | ADAC (μM) | | | | |
|---|---|---|---|---|---|
| siRNA | 0.063 μM | 0.013 μM | 0.25 μM | 0.51 μM | 1 μM |
| control | 15 | 19 | 35 | 43 | 54 |
| A1 | 16 | 18 | 37 | 41 | 52 |
| A2A | 6.7 | 12 | 15 | 19 | 24 |
| A2B | 12 | 17 | 34 | 40 | 53 |
| A3 | 18 | 22 | 41 | 46 | 54 |

We further evaluated the requirement for the A2A receptor by repeating the siRNA transfection and incubating cells with HE-NECA, a very potent A2A receptor at concentrations that are known to occupy/stimulate the A2A receptor fully (HE-NECA Kᵢ=˜27 nM). After siRNA transfection and at the time of HE-NECA addition to cells, A2A RNA levels were reduced >50% as determined by real time PCR. Again, silencing of the A2A receptor had a strong effect on adenosine receptor agonist activity (Table 35).

TABLE 35
Antiproliferative activity of potent adenosine receptor A2A agonist
HE-NECA against human multiple myeloma cells (MM.1R) after
transfection of siRNA silencing the adenosine A2A receptor subtype

| | HE-NECA (μM) | | | | |
|---|---|---|---|---|---|
| siRNA | 0.25 μM | 0.5 μM | 1 μM | 2 μM | 4.1 μM |
| control | 67 | 68 | 68 | 73 | 74 |
| A2A | 24 | 30 | 29 | 38 | 40 |

### EXAMPLE 6

### Phosphodiesterase Inhibitor Analysis

To better understand the phosphodiesterase (PDE) target in MM cells, we have crossed a panel of PDE inhibitors with the adenosine receptor agonists chloro-IB-MECA, HE-NECA, (S)-ENBA, and/or ADAC in MM.1S or H929 cells. The PDE inhibitors that showed synergy (score>1) include BAY-60-7550 (PDE 2 inhibitor), cilostamide, cilostazol and milrinone (PDE 3 inhibitors), rolipram, R-(−)-rolipram, RO-20-1724 and roflumilast (PDE 4 inhibitors), trequinsin (PDE 2/PDE 3/PDE 4 inhibitor) and zardaverine (PDE 3/PDE 4 inhibitor) and papaverine and BRL-50481 (PDE 7 inhibitors). Factors that influenced the extent to which the various PDE inhibitors were active include their specificity and the extent to which they are cell permeable.

TABLE 36

| | PDE inhibitors (specificity) | | | | | | | |
|---|---|---|---|---|---|---|---|---|
| | Chloro-IB- | | HE- | | (S) | | | |
| | MECA | | NECA | | ENBA | | ADAC | |
| | MM.1S | H929 | MM.1S | H929 | MM.1S | H929 | MM.1S | H929 |
| IBMX (pan) | | | | | 0.055 | | | |
| Pentoxifylline (pan) | 0.05 | 0.02 | 0.29 | 0.09 | 0.49 | 0.02 | | |
| Sildenafil (1, 5) | 0 | 0.03 | 0 | 0 | 0 | 0.03 | 0 | 0.14 |
| Vinoceptine (1) | | | 0.26 | 0 | 0.25 | 0.21 | 0.02 | 0.01 |
| BAY 60-7550 (2) | 0.86 | 0.74 | 3.8 | 3.71 | 2.84 | 1.07 | 0.85 | 0.55 |
| Trequinsin (2, 3, 4) | 5.95 | 2.77 | 7.85 | 4.34 | 4.56 | 3.38 | 6.62 | 3.04 |
| Cilostamide (3) | 1.1 | 0.65 | 0.49 | 0.28 | | 1.17 | | |
| Cilostazol (3) | 0.75 | 0.46 | 3.50 | 1.21 | 1.73 | 0.4 | 0.89 | 0.36 |
| Milrinone (3) | 0.25 | 0.08 | 0.33 | 0.15 | 1.31 | | | |
| Siguazodan (3) | 0.42 | 0.09 | 0.72 | 0.08 | 1.39 | 0.13 | | |
| Ibudilast (3, 4, 10, 11) | 0.74 | 0.32 | 0.98 | 0.32 | 0.55 | 0.21 | 0.23 | 1.04 |
| Irsogladine (4) | 0.25 | 0.05 | | | 0.38 | 0.09 | | |
| (R)-Rolipram (4) | 0.84 | 0.63 | 4.38 | 2.51 | 2.08 | 0.82 | 0.97 | 0.51 |
| RO-20-1724 (4) | 1.6 | 1.14 | 3.58 | 2.51 | 0.73 | 0.09 | 1.71 | 1.11 |
| Zaprinast (1, 6, 10, 11) | 0.05 | 0.03 | 0.025 | 0.13 | 0.16 | 0.05 | | |
| Dipyridamole | 0.20 | 0.08 | 0.08 | 0.13 | 0.17 | 0.26 | 1.18 | 2.05 |
| (5, 6, 7, 8, 10, 11) | | | | | | | | |
| Papaverine (6, 7, 10) | 2.67 | 1.42 | 2.09 | 1.03 | 2.24 | 0.77 | 3.55 | 1.19 |
| Zardaverine (3, 4) | 3.71 | 2.97 | 4.39 | 3.39 | 2.59 | 4.02 | | |
| Roflumilast (4) | 2.12 | 1.16 | 2 | 0.98 | 2.19 | 1.77 | | |
| Rolipram (4) | 1.11 | 0.74 | 1.08 | 1.09 | 0.73 | 0.46 | 1.15 | 1.11 |
| BRL-50481(7) | 1.47 | 0.34 | 1.41 | 0.23 | 1.22 | 0.26 | | |

We examined the activity of PDE inhibitors when used in combination with adenosine receptor agonist using additional multiple myeloma cell lines to examine the breadth of activity of this type of combination on MM cell growth. As shown in Table 37, adenosine receptor agonist/PDE combinations were synergistically antiproliferative in almost all of the cell lines examined, with more activity observed with PDE 3/4 inhibitors than PDE 4 inhibitors, consistent with the inhibition of multiple PDEs for maximal activity.

TABLE 37
Summary of synergy scores for adenosine receptor agonist
CGS-21680 × PDE inhibitors in the MOLP-8, EJM, INA-6,
ANBL6, KSM-12-PE, and OPM2 MM cell lines.

| | | | | | KSM- | |
|---|---|---|---|---|---|---|
| | MOLP-8 | EJM | INA-6 | ANBL6 | 12-PE | OPM2 |
| roflumilast | 3.44 | 1.06 | 2.62 | 3.73 | 0.27 | 0.29 |
| trequinsin | 4.7 | 4.81 | 3.93 | 4.55 | 2.44 | 4.74 |
| zardaverine | 3.06 | 0.98 | 2.69 | 2.11 | 0.49 | 1.15 |

Of all the PDE inhibitors, trequinsin and zardaverine (both PDE 3/PDE 4 inhibitors) had the highest synergy scores when crossed with adenosine receptor agonists. As PDE 2, PDE 3, and PDE 4 inhibitors were not as potent as either trequinsin or zardaverine, we performed crosses using mixtures of PDE inhibitors (PDE 2 with PDE 3, PDE 3 with PDE 4 and PDE 2 with PDE 4 (Table 38)) to determine if the use of inhibitors that targeted individual PDEs would show an increase in activity if used in combination.

Crosses (6×6) were performed between PDE inhibitors (PDEi) and HE-NECA. For the PDE mixtures, the relative concentrations were BAY 60-7550/R-(−)-rolipram at a ratio of 1.9:1, BAY 60-7550/cilostazol at a ratio of 1.5:1 and cilostazol/R-(−)-rolipram at a ratio of 3:1. In each case, the synergy observed for the PDE mixtures was higher than for the individual compounds, suggesting that for maximal synergistic antiproliferative effect, the PDE targets include PDE 2, PDE 3, PDE 4, and PDE 7 (identified using papaverine and BRL-50481).

TABLE 38

| PDEi × HE-NECA | MM.1S | H929 |
|---|---|---|
| BAY 60-7550 | 1.64 | 1.68 |
| Cilostamide | 1.02 | 0.56 |
| R-(−)-Rolipram | 2.33 | 1.88 |
| Trequinsin | 5.7 | 4.22 |
| BAY 60-7550 + | 3.27 | 2.13 |
| Cilostamide | | |
| BAY 60-7550 + R-(−)- | 2.85 | 2.53 |
| Rolipram | | |
| Cilostamide + R-(−)- | 3.41 | 2.65 |
| Rolipram | | |
| Zardaverine | 4.39 | 3.39 |

We have examined the antiproliferative activity of adenosine receptor agonists/PDE inhibitor combinations after MM.1R is transfected with siRNA targeting the PDE 2A, PDE 3B, PDE 4B, PDE 4D, or PDE 7A. As the chemical genetic analysis pointed to the importance of these four PDE family members, and all four act in cells to reduce the levels of cAMP, the effects of targeting one PDE would likely be subtle and increased if siRNA was used in concert with compounds that inhibit other family members or agents such as A2A agonists, that elevate the levels of cAMP in the cell.

In our experiments, PDE gene silencing was always greater than 50% as confirmed by real time PCR analysis 48 hours post-transfection. At 48 hours post-transfection, cells were exposed to adenosine receptor agonist and PDE inhibitor, incubated an additional 72 hours, and compounds assayed for antiproliferative activity. Representative data is in Tables 39-45. For each analysis, the activity of cells transfected with an siRNA targeting a specific PDE was compared to cells transfected with a control non-targeting siRNA (siCON). As seen in Tables 39 and 40, transfection of cells with an siRNA targeting PDE 3B increased the activity of the drug combination HE-NECA and roflumilast (a PDE 4 inhibitor). At the time of drug combination addition, PDE 3B RNA levels had been reduced 64% as determined by real time PCR.

TABLE 39
Antiproliferative activity of HE-NECA and roflumilast against human
multiple myeloma cells (MM.1R) after transfection with control
(non-targeting) siRNA (siCON).

HE-NECA (nM)

| Roflumilast (μM) | 20 | 6.8 | 2.3 | 0.75 | 0.25 | 0 |
|---|---|---|---|---|---|---|
| 1.0 | 70 | 76 | 70 | 56 | 31 | 14 |
| 0.50 | 80 | 82 | 69 | 57 | 25 | 8.7 |
| 0.25 | 78 | 79 | 69 | 49 | 30 | 3.5 |
| 0.13 | 83 | 76 | 70 | 49 | 22 | 0.3 |
| 0.063 | 76 | 73 | 66 | 42 | 25 | −8 |
| 0 | 64 | 54 | 40 | 17 | 20 | −7.4 |

TABLE 40
Antiproliferative activity of HE-NECA and roflumilast against human
multiple myeloma cells (MM.1R) after transfection with PDE 3B
siRNA

HE-NECA (nM)

| Roflumilast (μM) | 20 | 6.8 | 2.3 | 0.75 | 0.25 | 0 |
|---|---|---|---|---|---|---|
| 1.0 | 83 | 86 | 79 | 70 | 54 | 18 |
| 0.50 | 88 | 84 | 82 | 74 | 46 | 10 |
| 0.25 | 86 | 86 | 81 | 70 | 46 | 6.8 |
| 0.13 | 88 | 83 | 81 | 71 | 49 | 11 |
| 0.063 | 88 | 86 | 80 | 70 | 48 | 3 |
| 0 | 66 | 59 | 50 | 27 | 12 | −3.7 |

Shown in Tables 41 and 42 is the effect on drug combination activity (HE-NECA×cilostazol, a PDE 3 inhibitor) when cells were transfected with siRNA to PDE 7A (PDE 7A RNA reduced 60% at the time of drug addition).

TABLE 41
Antiproliferative activity of HE-NECA and cilostazol against human
multiple myeloma cells (MM.1R) after transfection with control
(non-targeting) siRNA

HE-NECA (nM)

| Cilostazol (μM) | 20 | 6.8 | 2.3 | 0.75 | 0.25 | 0 |
|---|---|---|---|---|---|---|
| 27 | 84 | 80 | 77 | 65 | 57 | 31 |
| 9.0 | 80 | 69 | 67 | 48 | 34 | 4.7 |
| 3.0 | 71 | 70 | 61 | 43 | 24 | −7.5 |
| 1.0 | 69 | 66 | 52 | 34 | 23 | 1.6 |
| 0.34 | 66 | 62 | 43 | 32 | 20 | −2.5 |
| 0 | 63 | 55 | 48 | 19 | 27 | −9.7 |

TABLE 42
Antiproliferative activity of HE-NECA and cilostazol against human
multiple myeloma cells (MM.1R) after transfection with PDE 7A
siRNA

HE-NECA (nM)

| Cilostazol (μM) | 20 | 6.8 | 2.3 | 0.75 | 0.25 | 0 |
|---|---|---|---|---|---|---|
| 27 | 87 | 87 | 82 | 78 | 60 | 36 |
| 9.0 | 83 | 78 | 77 | 61 | 40 | 6.1 |
| 3.0 | 78 | 77 | 63 | 54 | 18 | 7.7 |
| 1.0 | 78 | 70 | 66 | 43 | 27 | −8.6 |
| 0.34 | 73 | 69 | 55 | 45 | 12 | −2.5 |
| 0 | 71 | 65 | 56 | 33 | 17 | −8.5 |

Shown in Tables 43-45 is the effect on drug combination activity (HE-NECA×BAY 60-7550, a PDE 2 inhibitor) when cells were transfected with siRNA to PDE 4B (PDE 4B RNA reduced 54% at the time of drug addition) or PDE 4D (PDE 4D RNA reduced 57%).

TABLE 43
Antiproliferative Activity of HE-NECA and BAY 60-7550 Against
Human Multiple Myeloma cells (MM.1R) after Transfection with
Control (Non-targeting) siRNA

| | HE-NECA (nM) | | | | | |
|---|---|---|---|---|---|---|
| BAY 60-7550 (μM) | 20 | 6.8 | 2.3 | 0.75 | 0.25 | 0 |
| 35 | 91 | 88 | 84 | 71 | 50 | 5.9 |
| 12 | 85 | 81 | 72 | 58 | 35 | 6.8 |
| 4 | 78 | 74 | 66 | 45 | 20 | 2.8 |
| 1.3 | 72 | 63 | 54 | 44 | 24 | 2 |
| 0.44 | 70 | 59 | 52 | 28 | 9 | −8.1 |
| 0 | 60 | 53 | 44 | 26 | 6.1 | −0.2 |

TABLE 44
Antiproliferative Activity of HE-NECA and BAY 60-7550 Against
Human Multiple Myeloma cells (MM.1R) after Transfection with
PDE 4B siRNA

| | HE-NECA (nM) | | | | | |
|---|---|---|---|---|---|---|
| BAY 60-7550 (μM) | 20 | 6.8 | 2.3 | 0.75 | 0.25 | 0 |
| 35 | 94 | 89 | 88 | 75 | 53 | 15 |
| 12 | 88 | 84 | 79 | 68 | 32 | 1.6 |
| 4 | 82 | 77 | 74 | 52 | 26 | −0.8 |
| 1.3 | 78 | 73 | 63 | 48 | 26 | 8.7 |
| 0.44 | 74 | 62 | 58 | 31 | 16 | 2.3 |
| 0 | 74 | 66 | 53 | 35 | 3.3 | 0.2 |

TABLE 45
Antiproliferative Activity of HE-NECA and BAY 60-7550 Against
Human Multiple Myeloma cells (MM.1R) after Transfection with
PDE 4D siRNA

| | HE-NECA (nM) | | | | | |
|---|---|---|---|---|---|---|
| BAY 60-7550 (μM) | 20 | 6.8 | 2.3 | 0.75 | 0.25 | 0 |
| 35 | 93 | 87 | 86 | 74 | 48 | 22 |
| 12 | 86 | 84 | 77 | 67 | 38 | 13 |
| 4 | 81 | 77 | 73 | 49 | 28 | 10 |
| 1.3 | 75 | 72 | 60 | 49 | 20 | 7.7 |
| 0.44 | 70 | 61 | 58 | 26 | 11 | −7.5 |
| 0 | 71 | 62 | 54 | 42 | 7.6 | 5.4 |

Shown in Tables 46-47 is the effect on drug combination activity (HE-NECA×R-(−)-Rolipram, a PDE 4 inhibitor) when MM.1R cells were transfected with a control siRNA (non-targeting) or an siRNA targeting PDE 2A. Similar to what is seen when reducing the expression of PDE 3B, PDE 4B, PDE 4D, and PDE 7A, reducing the levels of PDE 2 increases the activity of the drug combination. The relatively modest effect on activity was likely due to the fact that the expression of the PDE targets was never knocked down 100% and that PDE activity is redundant (PDE 2, 3, 4 and 7 contributing to cAMP regulation).

TABLE 46
Antiproliferative activity of HE-NECA and R-(−)-rolipram against
human multiple myeloma cells (MM.1R) after transfection with
control (non-targeting) siRNA.

| | HE-NECA (nM) | | | | | |
|---|---|---|---|---|---|---|
| R-(−)-Rolipram (μM) | 20 | 10 | 5 | 2.5 | 1.25 | 0 |
| 18 | 78 | 72 | 74 | 74 | 66 | 8.9 |
| 6.1 | 82 | 75 | 74 | 64 | 68 | 5.2 |
| 2 | 81 | 71 | 71 | 68 | 71 | −2.4 |
| 0.68 | 78 | 72 | 68 | 66 | 65 | 3.5 |
| 0.23 | 72 | 66 | 66 | 40 | 49 | 7.6 |
| 0 | 57 | 51 | 41 | 41 | 43 | 2.2 |

TABLE 47
Antiproliferative activity of HE-NECA and R-(−)-rolipram against
human multiple myeloma cells (MM.1R) after transfection with
siRNA targeting PDE 2A.

| | HE-NECA (nM) | | | | | |
|---|---|---|---|---|---|---|
| R-(−)-Rolipram (μM) | 20 | 10 | 5 | 2.5 | 1.25 | 0 |
| 18 | 82 | 76 | 78 | 78 | 65 | 7.7 |
| 6.1 | 83 | 78 | 76 | 75 | 75 | 5.3 |
| 2 | 84 | 80 | 76 | 71 | 75 | 8.1 |
| 0.68 | 80 | 76 | 73 | 67 | 68 | −1.2 |
| 0.23 | 72 | 74 | 68 | 46 | 58 | 3.8 |
| 0 | 68 | 55 | 51 | 48 | 36 | −2.7 |

### EXAMPLE 7

### Activity in Other Cell Lines

The anti-proliferative activity of adenosine receptor agonists and PDE inhibitors was examined using the GA-10 (Burkitt's lymphoma) cell line. As with the multiple myeloma cell lines, synergy was observed when adenosine receptor agonists were used in combination with PDE inhibitors (Table 48). Similar results were obtained with the DLBCL cell lines OCI-ly10, Karpas 422, and SU-DHL6 (Table 49).

TABLE 48
Summary of synergy scores for adenosine receptor agonists × PDE
inhibitors in GA-10 cell line

| Adenosine receptor agonist (×) | |
|---|---|
| PDE inhibitor | GA-10 |
| Chloro-IB-MECA × BAY 60-7550 | 1.42 |
| CGS-21680 × BAY 60-7550 | 1.65 |
| Chloro-IB-MECA × Roflumilast | 0.56 |
| IB-MECA × Roflumilast | 0.95 |
| CGS-21680 × Roflumilast | 1.2 |

TABLE 49
Summary of synergy scores for adenosine receptor agonist
CGS-21680 × PDE inhibitors in the diffuse large B-cell
lymphoma cell lines OCI-ly10, Karpas 422, and SU-DHL6

| | OCI-ly10 | Karpas 422 | SU-DHL6 |
|---|---|---|---|
| CGS-21680 × Trequinsin | 1.64 | 2.11 | 0.92 |
| CGS-21680 × Roflumilast | 3.32 | 3.38 | 0.93 |

As there are no cell lines available for the B cell cancer chronic lymphocytic leukemia (CLL), tumor cells were isolated from a patient with the disease, and cells cultured in the presence of the adenosine receptor agonist CGS-21680 and either the PDE inhibitor roflumilast (Table 50) or the PDE 2/3/4 inhibitor trequinsin (Table 51). Combination (more than additive) induction of apoptosis was observed with both the CGS-21680× roflumilast and the CGS-21680× trequinsin combinations.

TABLE 50
Induction of apoptosis of patient CLL cells by CGS-21680 and
roflumilast

CGS-21680 (μM)

| Roflumilast (μM) | 0.45 | 0.15 | 0.05 | 0 |
|---|---|---|---|---|
| 0.27 | 46 | 45 | 43 | 32 |
| 0.09 | 38 | 40 | 36 | 26 |
| 0.03 | 34 | 35 | 31 | 17 |
| 0 | 25 | 15 | 12 | 5.9 |

TABLE 51
Induction of apoptosis of patient CLL cells by CGS-21680 and
trequinsin

CGS-21680 (μM)

| Trequinsin (μM) | 0.45 | 0.15 | 0.05 | 0 |
|---|---|---|---|---|
| 2 | 33 | 23 | 20 | 19 |
| 0.67 | 35 | 13 | 13 | 9.9 |
| 0.22 | 18 | 11 | 9.7 | 8.9 |
| 0 | 27 | 16 | 16 | 12 |

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

What is claimed is:

1. A method of treating a B-cell proliferative disorder, said method comprising administering to a patient a combination of an A2A receptor agonist and a PDE inhibitor in amounts that together are effective to treat said B-cell proliferative disorder.

2. The method of claim 1, wherein said A2A receptor agonist is selected from the group consisting of the compounds listed in Tables 1 and 2.

3. The method of claim 1, wherein said PDE inhibitor is selected from the group consisting of the compounds listed in Tables 3 and 4.

4. The method of claim 1, wherein said PDE inhibitor is active against at least two of PDE 2, 3, 4, and 7.

5. The method of claim 1, wherein said combination comprises two or more PDE inhibitors that when combined are active against at least two of PDE 2, 3, 4, and 7.

6. The method of claim 1, wherein said B-cell proliferative disorder is selected from the group consisting of autoimmune lymphoproliferative disease, B-cell CLL, B-cell prolymphocyte leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, indolent myeloma, smoldering myeloma, monoclonal gammopathy of unlcnown significance (MGUS), B-cell non-Hodgkin's lymphoma, small lymphocytic lymphoma, monoclonal immunoglobin deposition diseases, heavy chain diseases, mediastinal (thymic) large B-cell lymphoma, intravascular large B-cell lymphoma, primary effusion lymphoma, lymphomatoid granulomatosis, precursor B-lymphoblastic leukemia/lymphoma, Hodgkin's lymphoma, nodular lymphocyte predominant Hodgkin's lymphoma, classical Hodgkin's lymphoma, nodular sclerosis Hodgkin's lymphoma, mixed cellularity Hodgkin's lymphoma, lymphocyte-rich classical Hodgkin's lymphoma, lymphocyte depleted Hodgkin's lymphoma, post-transplant lymphoproliferative disorder, and Waldenstrom's macroglobulineamia.

7. The method of claim 1, wherein said B-cell proliferative disorder is multiple myeloma.

8. The method of claim 1, wherein said A2A receptor agonist and PDE inhibitor are administered simultaneously.

9. The method of claim 1, wherein said A2A receptor agonist and PDE inhibitor are administered within 14 days of one another.

10. The method of claim 1, wherein said patient is not suffering from a comorbid immunoinflammatory disorder.

11. The method of claim 1, further comprising administering an antiproliferative compound.

12. The method of claim 11, wherein said antiproliferative compound is selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors, CD40 inhibitors, anti-CSI antibodies, FGFR3 inhibitors, VEGF inhibitors, MEK inhibitors, cyclin D1 inhibitors, NF-kB inhibitors, anthracyclines, histone deacetylases, kinesin inhibitors, phosphatase inhibitors, COX2 inhibitors, mTOR inhibitors, calcineurin antagonists, and IMiDs.

13. The method of claim 11, wherein said antiproliferative compound is selected from the compounds listed in Tables 5 and 6.

14. The method of claim 1, further comprising administering a combination of at least two antiproliferative compounds.

15. The method of claim 14, wherein said combination is selected from the group consisting of CHOP (cyclophosphamide, vincristine, doxorubicin, and prednisone), VAD (vincristine, doxorubicin, and dexamethasone), MP (melphalan and prednisone), DT (dexamethasone and thalidomide), DM (dexamethasone and melphalan), DR (dexamethasone and Revlimid), DV (dexamethasone and Velcade), RV (Revlimid and Velcade), and cyclophosphamide and etoposide.

16. The method of claim 1, further comprising administering IL-6, a compound that increases IL-6 expression, or an IL-6 receptor agonist to said patient.

17. The method of claim 1, wherein said PDE inhibitor is active against PDE 4.

18. A kit comprising (i) a PDE inhibitor and (ii) an A2A receptor agonist in an amount effective to treat a B-cell proliferative disorder.

19. A kit comprising (i) an A2A receptor agonist and (ii) a PDE inhibitor having activity against at least two of PDE 2, 3, 4, and 7.

20. A kit comprising (i) an A2A receptor agonist and (ii) two or more PDE inhibitors that when combined have activity against at least two of PDE 2, 3, 4, and 7.

21. A kit comprising (i) an A2A receptor agonist, (ii) a PDE inhibitor, and (iii) an antiproliferative compound.

22. The kit of claim 18-20, further comprising an antiproliferative compound.

23. The kit of claim 21-22, wherein said antiproliferative compound is selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonist, retinoic acid receptor agonists, immuno-modulators, hormonal and antihormonal agents, photodynamic agents, tyrosine kinase inhibitors, antisense compounds, corticosteroids, HSP90 inhibitors, proteosome inhibitors, CD40 inhibitors, anti-CSI antibodies, FGFR3 inhibitors, VEGF inhibitors, MEK inhibitors, cyclin D1 inhibitors, NF-kB inhibitors, anthracyclines, histone deacetylases, kinesin inhibitors, phosphatase inhibitors, COX2 inhibitors, mTOR inhibitors, calcineurin antagonists, and IMiDs.

24. The kit of claims 21-22, further comprising at least a second antiproliferative compound in a combination with said antiproliferative compound.

25. The kit of claim 24, wherein said combination is selected from the group consisting of CHOP (cyclophosphamide, vincristine, doxorubicin, and prednisone), VAD (vincristine, doxorubicin, and dexamethasone), MP (melphalan and prednisone), DT (dexamethasone and thalidomide), DM (dexamethasone and melphalan), DR (dexamethasone and Revlimid), DV (dexamethasone and Velcade), RV (Revlimid and Velcade), and cyclophosphamide and etoposide.

26. A pharmaceutical composition comprising (i) a PDE inhibitor and (ii) an A2A receptor agonist in an amount effective to treat a B-cell proliferative disorder and (iii) a pharmaceutically acceptable carrier.

27. A pharmaceutical composition comprising (i) an A2A receptor agonist and (ii) a PDE inhibitor having activity against at least two of PDE 2, 3, 4, and 7 and (iii) a pharmaceutically acceptable carrier.

28. A pharmaceutical composition comprising (i) an A2A receptor agonist and (ii) two or more PDE inhibitors that when combined have activity against at least two of PDE 2, 3, 4, and 7 and (iii) a pharmaceutically acceptable carrier.

29. A kit comprising:
(i) a composition comprising an A2A receptor agonist and a PDE inhibitor; and
(ii) instructions for administering said composition to a patient for the treatment of a B-cell proliferative disorder.

30. A kit comprising:
(i) an A2A receptor agonist; and
(ii) instructions for administering said A2A receptor agonist with a PDE inhibitor to a patient for the treatment of a B-cell proliferative disorder.

31. A kit comprising:
(i) a PDE inhibitor; and
(ii) instructions for administering said PDE inhibitor with an A2A receptor agonist to a patient for the treatment of a B-cell proliferative disorder.

32. A kit comprising:
(i) a PDE inhibitor;
(ii) an A2A receptor agonist; and
(iii) instructions for administering said PDE inhibitor and said A2A receptor agonist to a patient for the treatment of a B-cell proliferative disorder.

33. The kit of any of claims 29-32, wherein said PDE inhibitor has activity against at least two of PDE 2, 3, 4, and 7.

34. A kit comprising:
(i) two or more PDE inhibitors that when combined have activity against at least two of PDE2, 3, 4, and 7;
(ii) an A2A receptor agonist; and
(iii) instructions for administering said two or more PDE inhibitors and said A2A receptor agonist to a patient for the treatment of a B-cell proliferative disorder.
